(19) United States
(12) Patent Application Publication
(10) Pub. No.: US 2007/0060618 A1
(43) Pub. Date: Mar. 15, 2007

(54) Alkyne derivatives as tracers for metabotropic glutamate receptor binding

(75) Inventors: Nicholas David Peter Cosford, San Diego, CA (US); Steven Patrick Govck, Del Mar, CA (US); Terence Gerard Hamill, Lansdale, PA (US); Theodore Kamenecka, San Diego, CA (US); Thomas Jonathan Seiders, San Diego, CA (US); Jeffrey Roger Roppe, Temecula, CA (US); Celine Bonnefous, San Diego, CA (US); Donald Burns, Harleysville, PA (US); Matthew P. Braun, Scotch Plains, NJ (US); Dennis C. Dean, Cranford, NJ (US); Joseph Paul Simeone, Harrington Park, NJ (US)

Correspondence Address: MERCK AND CO., INC, P O BOX 2000, RAHWAY, NJ, 07065-0907, US

(73) Assignee: MERCK & CO., INC., RAHWAY, NJ (US)

(21) Appl. No.: 10/532,634

(22) PCT Filed: Oct. 24, 2003
(86) PCT No.: PCT/US03/33613

§ 371 Date: Apr. 25, 2005

Related U.S. Application Data
(60) Provisional application No. 60/420,809, filed on Oct. 24, 2002.

(51) Int. Cl. A61K 31/444; A61K 31/4439; A61K 31/426; A61K 51/00; C07D 417/14

(52) U.S. Cl. 514/332; 514/342; 514/369; 546/255; 546/256; 546/269.7; 548/201

(57) ABSTRACT

The present invention is directed to isotopically labeled alkyne derivative compounds, particularly ¹¹C, ¹³C, ¹⁴C, ¹⁸F, ¹⁵O, ¹³N, ³⁵S, ²H, and ³H labeled compounds. In particular, the present invention is directed to ¹¹C, ¹³C, ¹⁴C, ¹⁸F, ¹⁵O, ¹³N, ³⁵S, ²H, and ³H labeled heterocyclic alkynes and methods of their preparation. The present invention further includes a method of use of the ¹¹C, ¹⁸F, ¹⁵O, or ¹³N labeled heterocyclic alkyne compounds as tracers in positron emission tomography (PET) imaging, particularly in the study of metabolic conditions in mammals, specifically conditions modulated by metabotropic glutamate receptors subtype 5 (mGluR5).

### FIELD OF THE INVENTION

The present invention is directed to ¹¹C, ¹³C, ¹⁴C, ¹⁸F, 15O, ¹³N, ³⁵S, ²H, and ³H isotopically labeled heterocyclic alkyne derivative compounds. In particular, the present invention is directed to ¹¹C, ¹³C, ¹⁴C, ¹⁸F, ¹⁵O, ¹³N, ³⁵S, ²H, and ³H isotopes of heterocyclic alkynes and methods of their preparation.

The present invention further includes a method of use of the ¹¹C, ¹³C, ¹⁴C, ¹⁸F, ¹⁵O, ¹³N, ³⁵S, ²H, and ³H labeled heterocyclic alkyne compounds as tracers in positron emission tomography (PET) imaging and/or other forms of imaging for the study of metabolic conditions in mammals, specifically conditions modulated by metabotropic glutamate receptor subtype 5 (mGluR5).

### BACKGROUND OF THE INVENTION

Positron emission tomography (PET) is a type of nuclear imaging used in a variety of applications, particularly in medical research and diagnostic techniques. In a typical PET system, a radioactive compound, for example fluorodeoxyglucose (a radiopharmaceutical commonly referred to as FDG), is administered to the patient being tested. The isotopic compound then labels a selected substance that circulates in the blood of the patient and may be absorbed in certain tissues. Using available means of detection of positron emission, the radioisotope compound or tracer is then viewed in the various penetrated tissues in the body. Hence, PET imaging is a fast scanning technique for the study of various biological processes in vivo. For instance, PET provides the ability to study neurological diseases and disorders, including stroke, Alzheimer's disease, Parkinson's disease, epilepsy and cerebral tumors. Moreover, PET gives pharmaceutical research investigators the capability to assess biochemical changes or metabolic effects of a drug candidate in vivo for extended periods of time. Importantly, PET can measure drug distribution, thus allowing the evaluation of the pharmacokinetics and pharmacodynamics of a particular drug candidate under study. Consequently, interest in PET tracers for drug development has been expanding based on the development of isotopically labeled biochemicals and appropriate detection devices to detect the radioactivity by external imaging.

The isotopes used in PET tracer systems decay by emitting a positively charged particle with the same mass as the electron (a positron) and a neutrino from the nucleus. In this process one of the protons in the nucleus becomes a neutron, so that the isotope's atomic number declines while its atomic weight remains constant. The positron is ejected with a kinetic energy of up to 2 MeV, depending on the isotope, and loses this energy by collisions as it travels within the body of the patient. When the positron reaches a thermal energy level, it interacts with an electron, resulting in mutual annihilation of the two particles. The rest mass of the two particles is then transformed into two gamma rays of 511 KeV, which are characteristically emitted at 180° with respect to each other.

These two gamma rays may be detected by suitable devices. The devices are normally scintillation detectors arranged in a precise geometrical pattern around the patient. A scintillation detector emits a light flash, with the intensity of the light proportional to the energy of the gamma ray, each time it absorbs gamma radiation. Although this gamma radiation may or may not have arisen from the mutual annihilation of the positron and the electron, computer correlation and tomography graph the relevant annihilation events in time and space.

A wide range of compounds is used in PET imaging. These compounds, specifically positron-emitting radionuclides, have short half-lives and high radiation energies compared with radioisotopes generally used in biomedical research. The main positron-emitting radionuclides used in PET include carbon-11 (half-life of 20 minutes), nitrogen-13 (half-life of 10 minutes), oxygen-15 (half-life of 2 minutes), and fluorine 18 (half-life of 110 minutes). Accordingly, compounds containing such isotopes may be potentially useful as PET tracers. The specific activities (Ci/mmol) of these radionuclides are high because they are made through a nuclear transformation; that is, one element is converted into another such that, except for trace contaminants, they are carrier free. The actual specific activities for the commonly used PET radionuclides, ¹⁸F and ¹¹C, are of the order of 1000 to 5000 Ci/mmol at the end of the transformation by, for example, cyclotron bombardment. Therefore, these radioactive probes are injected at tracer levels in nmoles. This nuclear diagnostic technique, based on the tracer principle, facilitates measuring biochemical in vivo data, including the biochemistry of easily saturated sites such as receptors, by external imaging. For example, receptor binding studies include these three major areas:

A. Determine the interaction of the drug with a desired binding site (e.g. receptor or enzyme). Use the potential drug itself isotopically labeled in such a way not to disturb the biochemical parameter to be studied. Use a radioligand with the desired properties and study potential drug candidate binding by competition.

B. Measure neurotransmitter concentration changes with the reversible receptor radioligand indirectly after administering the potential drug whose putative mode of action is through neurotransmitter release.

C. Measure enzyme inhibition indirectly by measuring neurotransmitter concentration.

Due to elements carbon-11, nitrogen-13, and oxygen-15 being in most, if not all, of the compounds that are consumed by the human body, PET is an appropriate technique to study the fate of these compounds in vivo. Tracers, which are compounds labeled with ¹¹C, ¹⁸F, ¹⁵O or ¹³N radionuclides, may be administered by injection or inhalation; the purpose being simply to enter the compound into the bloodstream. It is the short half-lives of the radionuclides in these tracers that allow large doses to be administered to a subject with only low radiation exposure, and enable studies to be repeatedly performed on the same subject. The ability to study an animal or human more than once allow each live specimen to serve as its own control (improving the statistical power of the study) and permits interventional strategies to be followed over time.

Consequently over the past several years, PET has been undergoing very rapid development, mainly due to the multitudes of new tracer substances available for human studies. Nevertheless, there remains a need for novel PET tracers.

While the primary use of the isotopically labeled compounds of this invention is in positron emission tomography, which is an in vivo analysis technique, certain of the isotopically labeled compounds can be used in other than PET analyses. In particular, ¹⁴C and ³H labeled compounds can be used in in vitro and in vivo methods for the determination of binding, receptor occupancy and metabolic studies including covalent labeling. In particular, various isotopically labeled compounds find utility in magnetic resonance imaging, autoradiography and other similar analytical tools.

Metabotropic glutamate receptors (“mGluR”) are G protein-coupled receptors that activate intracellular second messenger systems when bound to the excitatory amino acid L-glutamic acid (glutamate). The mGluRs are divided into three groups based on amino acid sequence homology, transduction mechanism and pharmacological properties, namely Group I, Group II, and Group III. Each group of receptors contains one or more subtypes of receptors. For instance, Group I includes metabotropic glutamate receptors 1 and 5 (mGluR1 and mGluR5).

The mGluR's are further characterized by seven putative transmembrane domains preceded by a large putative extracellular amino-terminal domain and followed by a large putative intracellular carboxy-terminal domain. The receptors are coupled to G-proteins and activate certain second messengers depending on the receptor group. Thus, for example, Group I mGluR's activate phospholipase C. Activation of the receptor results in the hydrolysis of membrane phosphatidylinositol (4,5)-diphosphate to diacylglycerol, which activates protein kinase C, and inositol triphosphate, which in turn activates the inositol triphosphate receptor to promote the release of intracellular calcium.

Anatomical, biochemical and eletrophysiological analyses suggest that mGluR's, activated by glutamate, are a major excitatory neurotransmitter receptor class in the mammalian central nervous system. [Nakanishi et al., Brain Research Reviews 26:230-235 (1998); Monaghan et al., Ann. Rev. Pharmacol. Toxicol. 29:365-402 (1980).] This extensive repertoire of functions of mGluRs, especially those related to pain, anxiety/depression, drug addiction and withdrawal, disorders of the basal ganglia, and mental retardation, has stimulated recent attempts to describe and define the mechanisms through which glutamate exerts its effects.

According to anatomical studies in mammalian nervous system, mGluR5 is weakly expressed in the cerebellum, while higher levels of expression are found in the striatum and cortex (Romano et al., (1995) J. Comp. Neurol., 355:455-469). In the hippocampus, mGluR5 appears widely distributed and is diffusely expressed.

Because of the physiological and pathological significance of excitatory amino acid receptors, particularly metabotropic glutamate receptors, there is a need to develop methods such as PET imaging which would facilitate the research of the brain and central nervous system and further the development of therapeutic drugs which would treat conditions modulated by these receptors. Thus, there is a need for novel PET tracers that bind to various metabotropic glutamate receptors.

### SUMMARY OF THE INVENTION

The present invention is directed towards isotopically labeled alkyne derivative compounds, particularly ¹¹C, ¹³C, ¹⁴C, ¹⁸F, ¹⁵O, ¹³N, ³⁵S, ²H, and ³H labeled compounds. In particular, the present invention is directed to ¹¹C, ¹³C, ¹⁴C, ¹⁸F, 15O, ¹³N, ³⁵S, ²H, and ³H labeled heterocyclic alkynes and methods of their preparation.

The present invention further includes a method of use of the ¹¹C, ¹³C, ¹⁴C, ¹⁸F, ¹⁵O, ¹³N, ³⁵S, ²H, and ³H labeled heterocyclic alkyne compounds as tracers in positron emission tomography (PET) imaging. In a preferred embodiment, the present invention would serve as potential isotopically labeled ligands for metabotropic glutamate receptors and facilitate the study of metabolic conditions in mammals, specifically conditions modulated by metabotropic glutamate receptor subtype 5 (mGluR5).

### DETAILED DESCRIPTION OF THE INVENTION

The present invention is directed to isotopically labeled alkyne derivative compounds, particularly ¹¹C, ¹³C, ¹⁴C, ¹⁸F, ¹⁵O, ¹³N, ³⁵S, ²H, and ³H isotopes of heterocyclic alkynes, which have been identified as potent ligands for metabotropic glutamate receptors subtype 5 (mGluR5). The present invention is comprised of a substituted, unsaturated five-, six-, or seven-membered heterocyclic ring that includes at least one nitrogen atom and at least one carbon atom. The ring of such compounds additionally includes three, four or five atoms independently selected from carbon, nitrogen, sulfur, and oxygen atoms. The heterocyclic ring has at least one substituent located at a ring position adjacent to a ring nitrogen atom. This mandatory substituent of the ring includes a moiety, linked to the heterocyclic ring via an alkynylene moiety.

The present invention is directed to a compound represented by Formula I: 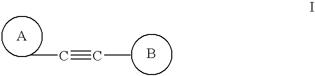
or a pharmaceutically acceptable salt thereof, wherein:

A is a heterocycle optionally substituted with one to five independent halogen, —CN, NO₂, —C₁₋₆alkyl, —C₁₋₆alkenyl, —C₁₋₆alkynyl, —OR¹, —NR¹R², —C(═NR¹)NR²R³, —N(═NR¹)NR²R³, —NR¹COR², —NR¹CO₂R², —NR¹SO₂R⁴, —NR¹CONR²R³, —SR⁴, —SOR⁴, —SO₂R⁴, —SO₂NR¹R², —COR¹, —CO₂R¹, —CONR¹R², —C(═NR¹)R², or —C(═NOR¹)R² substituents; wherein said alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R¹, R², and R³ each independently is —C₀₋₆alkyl, —C₃₋₇cycloalkyl, heteroaryl, or aryl; any of which is optionally substituted with 1-5 independent halogen, —N, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R⁴ is —C₁₋₆alkyl, —C₃₋₇cycloalkyl, heteroaryl, or aryl; optionally substituted with 1-5 independent halogen, —N, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

B is aryl, heterocycle, —C₃₋₂₀cycloalkyl, —C₃₋₂₀cycloalkenyl, —C₃₋₂₀cycloalkadienyl; —C₃₋₂₀cycloalkatrienyl, —C₃₋₂₀cycloalkynyl, —C₃₋₂₀cycloalkadiynyl, optionally substituted with one to five independent halogen, —CN, NO₂, —C₁₋₆alkyl, —C₁₋₆alkenyl, —C₁₋₆alkynyl, —OR₅, —NR⁵R⁶, —C(═NR⁵)NR⁶R⁷, —N(═NR⁵)NR⁶R⁷, —NR⁵COR⁶, —NR⁵CO₂R⁶, —NR⁵SO₂R⁸, —NR⁵CONR⁶R⁷, —SR⁸, —SOR⁸, —SO₂R⁸, —SO₂NR⁵R⁶, —COR⁵, —CO₂R⁵, —CONR⁵R⁶, —C(═NR⁵)R⁶, —C(═NOR⁵)R⁶, aryl or heterocycle substituents; wherein the alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆allyl)(C₀₋₆alkyl), —N(C₀₋₆alkyl)(C₃₋₇cycloalkyl), —N(C₀₋₆alkyl)(aryl) substituents;

R⁵, R⁶, and R⁷ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R⁸ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

wherein the compound is isotopically labeled with at least one ¹¹C, ¹³C, ¹⁴C, ¹⁸F, ¹⁵O, ¹³N, ³⁵S, ²H, or ³H atom;

and except when A=6-methyl-2-pyridyl then B cannot be 3-methoxyphenyl or unsubstituted phenyl.

Further compounds of this invention are represented by the compounds of Formula II. 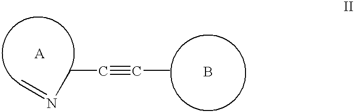
or a pharmaceutically acceptable salt thereof, wherein:

A is pyridinyl, pyrrolyl, imidazolyl, pyridazinyl, pyrimidinyl, pyrazoyl, pyrazinyl, triazolyl, triazinyl, tetrazolyl, tetrazinyl, tetrazepinyl, isoxazolyl, oxazolyl, oxadiazolyl, oxatriazolyl, oxazinyl, oxadiazinyl, isothiazolyl, thiazolyl, thiadazinyl, thiadiazolyl, thiadiazepinyl, dioxazolyl, oxathiazolyl, oxathiazinyl, oxazepinyl, oxadiazepinyl, azepinyl, and diazepinyl, optionally substituted with one to five independent halogen, —CN, NO₂, —C₁₋₆allyl, —C₁₋₆alkenyl, —C₁₋₆alkynyl, —OR¹, —NR¹R², —C(═NR¹)NR²R³, —N(═NR¹)NR²R³, —NR¹COR², —NR¹CO₂R², —NR¹SO₂R⁴, —NR¹CONR²R³, —SR⁴, —SOR⁴, —SO₂R⁴, —SO₂NR¹R², —COR¹, —CO₂R¹, —CONR¹R², —C(═NR¹)R², or —C(═NOR¹)R² substituents; wherein the alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R¹, R², and R³ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R⁴ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

B is phenyl, —C₃₋₂₀cycloalkyl, —C₃₋₂₀cycloalkenyl, —C₃₋₂₀cycloalkadienyl, —C₃₋₂₀cycloalkatrienyl, —C₃₋₂₀cycloalkynyl, —C₃₋₂₀cycloalkadiynyl, indenyl, dihydroindenyl, naphthalenyl, dihydronaphthalenyl, pyridinyl, thiazolyl, furyl, dihydropyranyl, dihydrothiopyranyl, piperidinyl, isoxazolyl, pyridazinyl, pyrimidinyl, pyrazinyl, indolyl, quinolinyl, isoquinolinyl, optionally substituted with one to five independent halogen, —CN, NO₂, —C₁₋₆alkyl, —C₁₋₆alkenyl, —C₁₋₆alkynyl, —OR⁵, —NR⁵R⁶, —C(═NR⁵)NR⁶R⁷, —N(═NR⁵)NR⁶R⁷, —NR⁵COR⁶, —NR⁵CO₂R⁶, —NR⁵SO₂R⁸, —NR⁵CONR⁶R⁷, —SR⁸, —SOR⁸, —SO₂R⁸, —SO₂NR⁵R⁶, —COR⁵, —CO₂R⁵, —CONR⁵R⁶, —C(═NR⁵)R⁶, —C(═NOR⁵)R⁶, aryl or heterocycle substituents; wherein the alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R⁵, R⁶, and R⁷ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R⁸ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

wherein the compound is isotopically labeled with at least one ¹¹C, ¹³C, ¹⁴C, ¹⁸F, ¹⁵O, ¹³N, ³⁵S, ²H, or ³H atom;

and except when A=6-methyl-2-pyridyl then B cannot be 3-methoxyphenyl or unsubstituted phenyl.

Unlabeled compounds analogous to the compounds described by Formula-I, and methods of their use, are disclosed in International Patent Publication No. WO 01/16121 A1.

In one aspect, the present invention is directed to a compound represented by Formula I, or a pharmaceutically acceptable salt, wherein:

A is pyridinyl, pyrrolyl, imidazolyl, pyridazinyl, pyrimidinyl, pyrazoyl, pyrazinyl, triazolyl, triazinyl, tetrazolyl, tetrazinyl, tetrazepinyl, isoxazolyl, oxazolyl, oxadiazolyl, oxatriazolyl, oxazinyl, oxadiazinyl, isothiazolyl, thiazolyl, thiadazinyl, thiadiazolyl, thiadiazepinyl, dioxazolyl, oxathiazolyl, oxathiazinyl, oxazepinyl, oxadiazepinyl, azepinyl, and diazepinyl, optionally substituted with one to five independent halogen, —CN, NO₂, —C₁₋₆alkyl, —C₁₋₆alkenyl, —C₁₋₆alkynyl, —OR¹, —NR¹R², —C(═NR¹)NR²R³, —N(═NR¹)NR²R³, —NR¹COR², —NR¹CO₂R², —NR¹SO₂R⁴, —NR¹CONR²R³, —SR⁴, —SOR⁴, —SO₂R⁴, —SO₂NR¹R², —COR¹, —CO₂R¹, —CONR¹R², —C(═NR¹)R², or —C(═NOR¹)R² substituents; wherein the alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R¹, R², and R³ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R⁴ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

B is phenyl, —C₃₋₂₀cycloalkyl, —C₃₋₂₀cycloalkenyl, —C₃₋₂₀cycloalkadienyl, —C₃₋₂₀cycloalkatrienyl, —C₃₋₂₀cycloalkynyl, —C₃₋₂₀cycloalkadiynyl, indenyl, dihydroindenyl, naphthalenyl, dihydronaphthalenyl, pyridinyl, thiazolyl, furyl, dihydropyranyl, dihydrothiopyranyl, piperidinyl, isoxazolyl, pyridazinyl, pyrimidinyl, pyrazinyl, indolyl, quinolinyl, isoquinolinyl, optionally substituted with one to five independent halogen, —CN, NO₂, —C₁₋₆alkyl, —C₁₋₆alkenyl, —C₁₋₆alkynyl, —OR⁵, —NR⁵R⁶, —C(═NR⁵)NR⁶R⁷, —N(═NR⁵)NR⁶R⁷, —NR⁵COR⁶, —NR⁵CO₂R⁶, —NR⁵SO₂R⁸, —NR⁵CONR⁶R⁷, —SR⁸, SOR⁸, ——SO₂R⁸, —SO₂NR⁵R⁶, —COR⁵, —CO₂R⁵, —CONR⁵R⁶, —C(═NR⁵)R⁶, —C(═NOR⁵)R⁶, aryl or heterocycle substituents; wherein the alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R⁵, R⁶, and R⁷ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R⁸ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

wherein the compound is isotopically labeled with at least one ¹¹C, ¹³C, ¹⁴C, ¹⁸F, ¹⁵O, ³N, ³⁵S, ²H, or ³H atom;

and except when A=6-methyl-2-pyridyl then B cannot be 3-methoxyphenyl or unsubstituted phenyl.

In a second aspect, the present invention is directed to a compound represented by Formula I, or a pharmaceutically acceptable salt, wherein:

A is thiazolyl or isothiazolyl, optionally substituted with one to three independent halogen, —CN, NO₂, —C₁₋₆alkyl, —C₁₋₆alkenyl, —C₁₋₆alkynyl, —OR^{l}, —NR¹R², —C(═NR¹)NR²R³, —N(═NR¹)NR²R³, —NR¹COR², —NR¹CO₂R², —NR¹SO₂R⁴, —NR¹CONR²R³, —SR⁴, —SOR⁴, —SO₂R⁴, —SO₂NR¹R², —COR¹, —CO₂R¹, —CONR¹R², —C(═NR¹)R², or —C(═NOR¹)R² substituents; and

B is phenyl, —C₃₋₂₀cycloalkyl, —C₃₋₂₀cycloalkenyl, —C₃₋₂₀cycloalkadienyl, —C₃₋₂₀cycloalkatrienyl, —C₃₋₂₀cycloalkynyl, —C₃₋₂₀cycloalkadiynyl, indenyl, dihydroindenyl, naphthalenyl, dihydronaphthalenyl, pyridinyl, thiazolyl, furyl, dihydropyranyl, dihydrothiopyranyl, piperidinyl, isoxazolyl, pyridazinyl, pyrimidinyl, pyrazinyl, indolyl, quinolinyl, isoquinolinyl, optionally substituted with one to five independent halogen, —CN, NO₂, —C₁₋₆alkyl, —C₁₋₆alkenyl, —C₁₋₆alkynyl, —OR⁵, —NR⁵R⁶, —C(═NR⁵)NR⁶R⁷, —N(═NR⁵)NR⁶R⁷, —NR⁵COR⁶, —NR⁵CO₂R⁶, —NR⁵SO₂R⁸, —NR⁵CONR⁶R⁷, —SR⁸, —SOR⁸, —SO₂R⁸, —SO₂NR⁵R⁶, —COR⁵, —CO₂R⁵, —CONR⁵R⁶, —C(═NR⁵)R⁶, —C(═NOR⁵)R⁶, aryl or heterocycle substituents;
wherein the compound is isotopically labeled with at least one ¹¹C, ¹³C, ¹⁴C, ¹⁸F, ¹⁵O, ¹³N, ³⁵S, ²H, or ³H atom.

In a third aspect, the present invention is directed to a compound represented by Formula I, or a pharmaceutically acceptable salt, wherein:

A is pyridinyl, pyrrolyl, imidazolyl, pyridazinyl, pyrimidinyl, pyrazoyl, pyrazinyl, triazolyl, triazinyl, tetrazolyl, tetrazinyl, tetrazepinyl, isoxazolyl, oxazolyl, oxadiazolyl, oxatriazolyl, oxazinyl, oxadiazinyl, isothiazolyl, thiazolyl, thiadazinyl, thiadiazolyl, thiadiazepinyl, dioxazolyl, oxathiazolyl, oxathiazinyl, oxazepinyl, oxadiazepinyl, azepinyl, and diazepinyl, optionally substituted with one to five independent halogen, —CN, NO₂, —C₁₋₆alkyl, —C₁₋₆alkenyl, —C₁₋₆alkynyl, —OR¹, —NR¹R², —C(═NR¹)NR²R³, —N(═NR¹)NR²R³, —NR¹COR², —NR¹CO₂R², —NR¹SO₂R⁴, —NR¹CONR²R³, —SR⁴, —SOR⁴, —SO₂R⁴, —SO₂NR¹R², —COR¹, —CO₂R¹, —CONR¹R², —C(═NR¹)R², or —C(═NOR¹)R² substituents;

R¹, R², and R³ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R⁴ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

B is pyridinyl or phenyl, optionally substituted with one to five independent halogen, —CN, NO₂, —C₁₋₆alkyl, —C₁₋₆alkenyl, —C₁₋₆alkynyl, —OR⁵, —NR⁵R⁶, —C(═NR⁵)NR⁶R⁷, —N(═NR⁵)NR⁶R⁷, —NR⁵COR⁶, —NR⁵CO₂R⁶, —NR⁵SO₂R⁸, —NR⁵CONR⁶R⁷, —SR⁸, —SOR⁸, —SO₂R⁸, —SO₂NR⁵R⁶, —COR⁵, —CO₂R⁵, —CONR⁵R⁶, —C(═NR⁵)R⁶, —C(═NOR⁵)R⁶, aryl or heterocycle substituents;

R⁵, R⁶, and R⁷ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

R⁸ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;

wherein the compound is isotopically labeled with at least one ¹¹C, ¹³C, ¹⁴C, ¹⁸F, ¹⁵O, ¹³N, ³⁵S, ²H, or ³H atom;

and except when A=6-methyl-2-pyridyl then B cannot be 3-methoxyphenyl or unsubstituted phenyl.

Preferred moieties include those wherein A is isothiazol-3-yl(1,2-thiazol-3-yl), thiazol-4-yl(1,3-thiazol-4-yl) and thiazol-2-yl(1,3-thiazol-2-yl). Other preferred moieties include those wherein A is oxazol-2-yl, isoxazol-3-yl and oxazol-4-yl. In a preferred embodiment of the invention, A is 2-pyridinyl, 3-pyridinyl or 2-pyrrolyl. Other preferred moieties include those wherein A is 3-pyridazinyl(1,2-diazin-3-yl), pyrimidin-4-yl(1,3-diazin-4-yl), pyrazin-3-yl(1,4-diazin-3-yl), pyrimidin-2-yl(1,3-diazin-2-yl), 1,3-isodiazol-4-yl and 1,3-isodiazol-2-yl. Presently preferred moieties include those wherein A is 1,2,3-triazin-4-yl, 1,2,4-triazin-6-yl, 1,2,4-triazin-3-yl, 1,2,4-triazin-5-yl, 1,3,5-triazin-2-yl, 1,2,3-triazol-4-yl, 1,2,4-triazol-3-yl. Presently preferred moieties include those wherein A is tetrazolyl. Presently preferred moieties include those wherein A is 1,2,4-thiadiazol-3-yl, 1,2,3-thiadiazol-4-yl, 1,3,4-thiadiazol-2-yl, 1,2,5-thiadiazol-3-yl and 1,2,4-thiadiazol-5-yl. Presently preferred moieties include those wherein A is 1,2,4-oxadiazol-3-yl, 1,2,3-oxadiazol-4-yl, 1,3,4-oxadiazol-2-yl, 1,2,5-oxadiazol-3-yl and 1,2,4-oxadiazol-5-yl.

Further preferred compounds of the invention are those wherein B is a substituted or unsubstituted aryl, cycloalkyl, cycloalkenyl, cycloalkadienyl, cycloalkatrienyl, cycloalkynyl, cycloalkadiynyl, bicyclic hydrocarbon wherein two rings have two atoms in common, and the like. Especially preferred compounds are those wherein B is cycloalkyl and cycloalkenyl having in the range of 4 up to about 8 carbon atoms. Exemplary compounds include cyclopropanyl, cyclopentenyl and cyclohexenyl. Also especially preferred are bicyclic hydrocarbon moieties wherein two rings have two atoms in common; exemplary compounds include indenyl, dihydroindenyl, naphthalenyl and dihydronaphthalenyl.

Still further preferred compounds of the invention are those wherein B is a substituted or unsubstituted heterocycle, optionally containing one or more double bonds. Exemplary compounds include pyridinyl, thiazolyl, furyl, dihydropyranyl, dihydrothiopyranyl, piperidinyl, isoxazolyl, pyridazinyl, pyrimidinyl, pyrazinyl, and the like. Also preferred are compounds wherein B is substituted or unsubstituted aryl. Especially preferred compounds are those wherein substituents are aryl and heterocycle, optionally bearing further substituents as described herein, methyl, trifluoromethyl, cyclopropyl, alkoxy, halogen and cyano. Also preferred are compounds wherein B is a bicyclic heterocycle moiety wherein two rings have two atoms in common. Exemplary compounds include indolyl and isoquinolinyl.

As selected from aforementioned moieties, B is further optionally substituted with one to five independent halogen, —C₁₋₁₂alkyl, —N(C₀₋₁₂alkyl)(C₀₋₁₂alkyl), or —O(C₁₋₁₂alkyl) substituents; and at least A or B is substituted with a fluorine-18 or a carbon-11 isotope.

As employed herein, “hydrocarbyl” refers to straight or branched chain univalent and bivalent radicals derived from saturated or unsaturated moieties containing only carbon and hydrogen atoms, and having in the range of about 1 up to 12 carbon atoms, unless otherwise stated. Exemplary hydrocarbyl moieties include alkyl moieties, alkenyl moieties, dialkenyl moieties, trialkenyl moieties, alkynyl moieties, alkadiynal moieties, alkatriynal moieties, alkenyne moieties, alkadienyne moieties, alkenediyne moieties, and the like. The term “substituted hydrocarbyl” refers to hydrocarbyl moieties further bearing substituents as set forth above.

As employed herein, “alkyl” refers to straight or branched chain alkyl radicals having in the range of about 1 up to 12 carbon atoms; “substituted alkyl” refers to alkyl radicals further bearing one or more substituents such as hydroxy, alkoxy, mercapto, aryl, heterocycle, halogen, trifluoromethyl, pentafluoroethyl, cyano, cyanomethyl, nitro, amino, amide, amidine, amido, carboxyl, carboxamide, carbamate, ester, sulfonyl, sulfonamide, and the like.

As employed herein, “cyclohydrocarbyl” refers to cyclic (i.e., ring-containing) univalent radicals derived from saturated or unsaturated moieties containing only carbon and hydrogen atoms, and having in the range of about 3 up to 20 carbon atoms. Exemplary cyclohydrocarbyl moieties include cycloalkyl moieties, cycloalkenyl moieties, cycloalkadienyl moieties, cycloalkatrienyl moieties, cycloalkynyl moieties, cycloalkadiynyl moieties, spiro hydrocarbon moieties wherein two rings are joined by a single atom which is the only common member of the two rings (e.g., spiro[3.4]octanyl, and the like), bicyclic hydrocarbon moieties wherein two rings are joined and have two atoms in common (e.g., bicyclo[3.2.1]octane, bicyclo[2.2.1]kept-2-ene, norbornene, decalin), and the like. The term “substituted cyclohydrocarbyl” refers to cyclohydrocarbyl moieties further bearing one or more substituents as set forth above.

As employed herein, “cycloalkyl” refers to ring-containing alkyl radicals containing in the range of about 3 up to 20 carbon atoms, and “substituted cycloalkyl” refers to cycloalkyl radicals further bearing one or more substituents as set forth above.

As employed herein, “aryl” refers to mononuclear and polynuclear aromatic radicals having in the range of 6 up to 14 carbon atoms, and “substituted aryl” refers to aryl radicals further bearing one or more substituents as set forth above, for example, alkylaryl moieties.

As employed herein, “heterocycle” refers to ring-containing radicals having one or more heteroatoms (e.g., N, O, S) as part of the ring structure, and having in the range of 3 up to 20 atoms in the ring. Heterocyclic moieties may be saturated or unsaturated when optionally containing one or more double bonds, and may contain more than one ring. Heterocyclic moieties include, for example, monocyclic moieties such as imidazolyl moieties, pyrimidinyl moieties, isothiazolyl moieties, isoxazolyl moieties, moieties, and the like, and bicyclic heterocyclic moieties such as azabicycloalkanyl moieties, oxabicycloalkyl moieties, and the like. The term “substituted heterocycle” refers to heterocycles further bearing one or more substituents as set forth above.

As employed herein, “halogen” refers to fluoride, chloride, bromide or iodide.

The present invention further discloses a method of use of isotopically labeled alkyne derivatives as tracers in positron emission tomography (PET) imaging for the study of metabolic conditions in mammals, specifically conditions modulated by metabotropic glutamate receptors subtype 5 (mGluR5). The alkyne derivatives possess superior binding affinities for mGluR5-rich tissues, such as the cerebral region and central nervous system. In particular, ¹¹C- or ¹⁸F-labeled alkyne derivatives have potential use in measuring mGluR5 receptor activity by PET imaging.

In that the described alkyne derivatives have high affinity for binding mGluR5 receptors and may be labeled with detectable “tagging” molecules, rendering labeled mGluR5 receptors highly visible through positron emission topography (PET), the present invention also relates to reagents, radiopharmaceuticals and techniques in the field of molecular imaging.

The alkyne derivatives of the present invention are advantageously used in the imaging of mGluR5 receptors, for example, in the central nervous system and may therefore be useful in the diagnosis of mGluR5-receptor positive cancers. The development of such derivatives would represent a tremendous improvement in the quality of imaging techniques currently available, as well as improve the accuracy of PET scans.

An ultimate objective of the present invention is to provide a radiopharmaceutical agent, useful in PET imaging that has high specific radioactivity and high target tissue selectivity by virtue of its high affinity for the mGluR5 receptor. The tissue selectivity is capable of further enhancement by coupling this highly selective radiopharmaceutical with targeting agents, such as microparticles. This method in one embodiment comprises positioning the patent supine, administering a sufficient quantity of a ¹¹C- or ¹⁸F-labeled mGluR5 ligand to a mGluR5 receptor-rich tissue; performing an emission scan of the mGluR5 receptor-rich tissue, and obtaining a PET image of the tissue; and evaluating said PET image for the presence or absence of focally increased uptake of the isotopically labeled ligand in the tissue.

In accordance with the present invention, the most preferred method for imaging mGluR5 receptors in a patient, wherein an isotopically labeled heterocyclic alkyne derivative is employed as the imaging agent, comprises the following steps: the patient is placed in a supine position in the PET camera, a sufficient amount (about 10 mCi) of an isotopically labeled heterocyclic alkyne derivative is administered to the brain tissue of the patient. An emission scan of the cerebral region is performed. The technique for performing an emission scan of the chest is well known to those of skill in the art. PET techniques are described in Freeman et al., Freeman and Johnson's Clinical Radionuclide Imaging. 3rd. Ed. Vol. 1 (1984); Grune & Stratton, New York; Ennis et Q. Vascular Radionuclide Imaging: A Clinical Atlas, John Wiley & Sons, New York (1983).

The term “labeled tracer” refers to any molecule which can be used to follow or detect a defined activity in vivo, for example, a preferred tracer is one that accumulates in metabotropic glutamate receptor rich regions. Preferably, the labeled tracer is one that can be viewed in a whole animal, for example, by positron emission tomograph (PET) scanning. Suitable labels include, but are not limited to radioisotopes, fluorochromes, chemiluminescent compounds, dyes, and proteins, including enzymes.

The present invention also provides methods of determining in vivo activity of an enzyme or other molecule. More specifically, a tracer, which specifically tracks the targeted activity, is selected and labeled. In a preferred embodiment, the tracer tracks binding activity to mGluR5 receptors in the brain and central nervous system. The tracer provides the means to evaluate various neuronal processes, including fast excitatory synaptic transmission, regulation of neurotransmitter release, and long-term potentiation. The present invention gives researchers the means to study the biochemical mechanisms of pain, anxiety/depression, drug addiction and withdrawal, disorders of the basal ganglia, eating disorders, obesity, long-term depression, learning and memory, developmental synaptic plasticity, hypoxic-ischemic damage and neuronal cell death, epileptic seizures, visual processing, as well as the pathogenesis of several neurodegenerative disorders.

Means of detecting labels are well know to those skilled in the art. For example, isotopic labels may be detected using imaging techniques, photographic film or scintillation counters. In a preferred embodiment, the label is detected in vivo in the brain of the subject by imaging techniques, for example positron emission tomography (PET).

The labeled compound of the invention preferably contains at least one radionuclide as a label. Positron-emitting radionuclides are all candidates for usage. In the context of this invention the radionuclide is preferably selected from ¹¹C, ¹³C, ¹⁴C, ¹⁸F, ¹⁵O, ¹³N, ³⁵S, ²H, and ³H.

The tracer can be selected in accordance with the detection method chosen. Before conducting the method of the present invention, a diagnostically effective amount of a labeled or unlabeled compound of the invention is administered to a living body, including a human.

The diagnostically effective amount of the labeled or unlabeled compound of the invention to be administered before conducting the in-vivo method for the present invention is within a range of from 0.1 ng to 100 mg per kg body weight, preferably within a range of from 1 ng to 10 mg per kg body weight.

In accordance with another embodiment of the present invention, there are provided methods for the preparation of heterocyclic compounds as described above. For example, the heterocyclic compounds described above can be prepared using synthetic chemistry techniques well known in the art (see Comprehensive Heterocyclic Chemistry, Katritzky, A. R. and Rees, C. W. eds., Pergamon Press, Oxford, 1984) from a precursor of the substituted heterocycle of Formula 1 as outlined below. The isotopically labeled compounds of this invention are prepared by incorporating an isotope such as ¹¹C, ¹³C, ¹⁴C, ¹⁸F, ¹⁵O, ¹³N, ³⁵S, ²H, and ³H into the substrate molecule. This is accomplished by utilizing reagents that have had one or more of the atoms contained therein made radioactive by placing them in a source of radioactivity such as a nuclear reactor, a cyclotron and the like. Additionally many isotopically labeled reagents, such as ²H₂O; ³H₃CI, ¹⁴C₆H₅Br, ClCH₂¹⁴COCl and the like, are commercially available. The isotopically labeled reagents are then used in standard organic chemistry synthetic techniques to incorporate the isotope atom, or atoms, into a compound of Formula I as described below. In the following Schemes any of A, B or L where L=alkyne or alkene linker may contain an isotope such as ¹¹C, ¹³C, ¹⁴C, ¹⁸F, ¹⁵O, ¹³N, ³⁵S, ²H, and ³H.

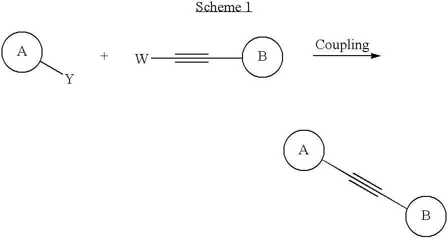

In Scheme 1, a substituted heterocycle precursor (prepared using synthetic chemistry techniques well known in the art) is reacted with an alkyne derivative. In Scheme 1, A and B are as defined above and Y and W are functional groups which ate capable of undergoing a transition metal-catalyzed cross-coupling reaction. For example, Y is a group such as hydrogen, halogen, acyloxy, fluorosulfonate, trifluoromethanesulfonate, alkyl- or arylsulfonate, alkyl- or arylsulfinate, alkyl- or arylsulfide, phosphate, phosphinate and the like, and W is hydrogen or a metallic or metalloid species such as Li, MgHal, SnR₃, B(OR)₂, SiR₃, GeR₃, and the like. The coupling may be promoted by a homogeneous catalyst such as PdCl₂(PPh₃)₂, or by a heterogeneous catalyst such as Pd on carbon in a suitable solvent (e.g. THF, DME, MeCN, DMF etc.). Typically a co-catalyst such as copper (I) iodide and the like and a base (e.g. NEt₃, K₂CO₃ etc.) will also be present in the reaction mixture. The coupling reaction is typically allowed to proceed by allowing the reaction temperature to warm slowly from about 0° C. up to ambient temperature over a period of several hours. The reaction mixture is then maintained at ambient temperature, or heated to a temperature anywhere between 30° C. 105° C. The reaction mixture is then maintained at a suitable temperature for a time in the range of about 4 up to 48 hours, with about 12 hours typically being sufficient. The product from the reaction can be isolated and purified employing standard techniques, such as solvent extraction, chromatography, crystallization, distillation and the like.

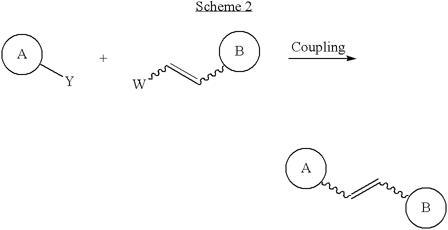

Another embodiment of the present invention is illustrated in Scheme 2. A substituted heterocycle precursor is reacted with an alkene derivative in a manner similar to the procedure described for Scheme 1. The product alkene derivative from Scheme 2 may be converted to an alkyne derivative using the approach outlined in Scheme 3.

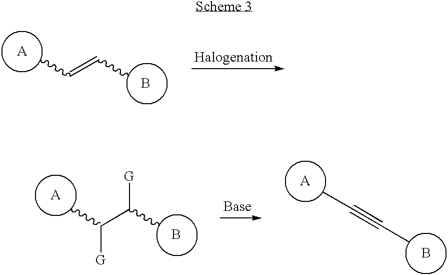

Thus, the alkene derivative may be contacted with a halogenating agent such as chlorine, bromine, iodine, NCS, NBS, NIS, ICl etc. in a suitable solvent (CCl₄, CHCl₃, CH₂Cl₂, AcOH and the like). The resulting halogenated derivative (G=halogen) is then treated with a suitable base such as NaOH, KOH, DBU, DBN, DABCO and the like which promotes double elimination reaction to afford the alkyne. The reaction is carried out in a suitable solvent such as EtOH, MeCN, toluene etc. at an appropriate temperature, usually between 0° C. and 150° C.

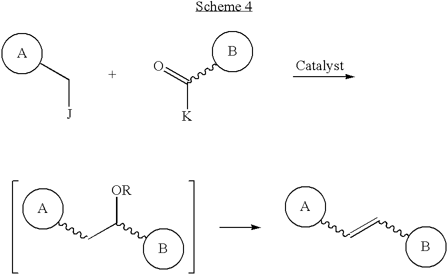

In another embodiment of the present invention, a substituted heterocyclic derivative is reacted with an aldehyde or ketone to provide a substituted alkene. Thus in Scheme 4, J is hydrogen, PR₃, P(O)(OR)₂, SO₂R, SiR₃ and the like, K is hydrogen, lower alkyl or aryl (as defined previously) and R is hydrogen, Ac and the like. Suitable catalysts for this reaction include bases such as NaH, nBuLi, LDA, LiHMDS, H₂NR, HNR₂, NR₃ etc., or electropositive reagents such as Ac₂O, ZnCl₂ and the like. The reaction is carried out in a suitable solvent (THF, MeCN etc.) at an appropriate temperature, usually between 0° C. and 150° C. Sometimes an intermediate is isolated and purified or partially purified before continuing through to the alkene product.

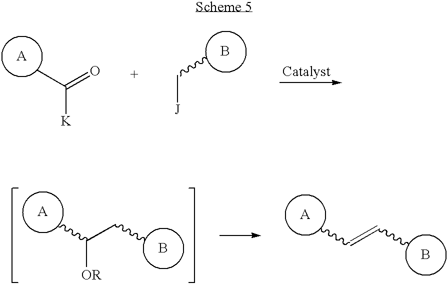

In yet another embodiment of the present invention, a substituted heterocyclic aldehyde or ketone is reacted with an activated methylene-containing compound to provide a substituted alkene. Thus in Scheme 5, J, K, R, the catalyst and reaction conditions are as described for Scheme 4. Again, as in Scheme 4, sometimes an intermediate is isolated and purified or partially purified before continuing through to the alkene product.

The alkene products from the reactions in Scheme 4 and Scheme 5 may be converted to an alkyne derivative using reagents and conditions as described for Scheme 3.

Another method for the preparation of heterocyclic compounds of Formula I is depicted in Scheme 6.

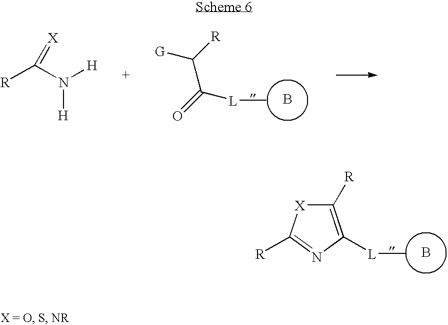

In Scheme 6, X may be O, S or NR and G is halogen or a similar leaving group, L is alkyne or alkene, B is as defined and R=substituents on A as previously described. The reagents are contacted in a suitable solvent such as EtOH, DMF and the like and stirred until the product forms. Typically reaction temperatures will be in the range of ambient through to about 150° C., and reaction times will be from 1 h to about 48 h, with 70° C. and 4 h being presently preferred. The heterocycle product can be isolated and purified employing standard techniques, such as solvent extraction, chromatography, crystallization, distillation and the like. Often, the product will be isolated as the hydrochloride or hydrobromide salt, and this material may be carried onto the next step with or without purification.

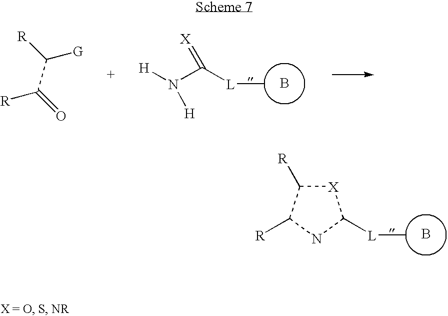

Yet another method for the preparation of heterocyclic compounds of Formula I is depicted in Scheme 7. In Scheme 7 X may be O, S or NR G is halogen or a similar leaving group, L is alkyne or alkene, B is as defined and R=substituents on A as previously described. The reaction conditions and purification procedures are as described for Scheme 6.

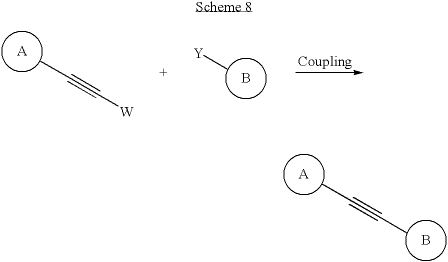

In another embodiment of the present invention, depicted in Scheme 8, an alkynyl-substituted heterocycle precursor (prepared using synthetic chemistry techniques well known in the art) is reacted with a species B, bearing a reactive functional group Y. In Scheme 8, A and B are as defined above and Y and W are functional groups which are capable of undergoing a transition metal-catalyzed cross-coupling reaction. For example, Y is a group such as hydrogen, halogen, acyloxy, fluorosulfonate, trifluoromethanesulfonate, alkyl- or arylsulfonate, alkyl- or arylsulfinate, alkyl- or arylsulfide, phosphate, phosphinate and the like, and W is hydrogen or a metallic or metalloid species such as Li, MgHal, SnR₃, B(OR)₂, SiR₃, GeR₃, and the like. The coupling may be promoted by a homogeneous catalyst such as PdCl₂(PPh₃)₂, or by a heterogeneous catalyst such as Pd on carbon in a suitable solvent (e.g. THF, DME, MeCN, DMF etc.). Typically a co-catalyst such as copper (I) iodide and the like and a base (e.g. NEt₃, K₂CO₃ etc.) will also be present in the reaction mixture. The coupling reaction is typically allowed to proceed by allowing the reaction temperature to warm slowly from about 0° C. up to ambient temperature over a period of several hours. The reaction mixture is then maintained at ambient temperature, or heated to a temperature anywhere between 30° C. to 150° C. The reaction mixture is then maintained at a suitable temperature for a time in the range of about 4 up to 48 hours, with about 12 hours typically being sufficient The product from the reaction can be isolated and purified employing standard techniques, such as solvent extraction, chromatography, crystallization, distillation and the like.

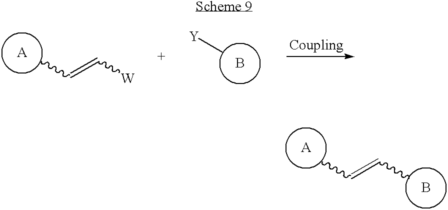

Another embodiment of the present invention is illustrated in Scheme 9. An alkenyl-substituted heterocycle precursor is reacted with an alkene derivative in a manner similar to the procedure described for Scheme 8. The product alkene derivative from Scheme 9 may be converted to an alkyne derivative using the approach outlined previously in Scheme 3 above.

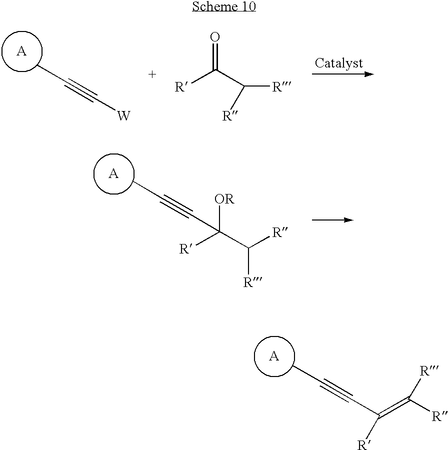

In yet another embodiment of the present invention, depicted in Scheme 10, an alkynyl-substituted heterocycle precursor is reacted with a species composed of a carbonyl group bearing substituents R′ and CHR″R′″. Thus in Scheme 10, R′, R″ and R′″ may be hydrogen or other substituents as described previously, or may optionally combine to form a ring (this portion of the molecule constitutes B in the final compound). W is hydrogen or a metallic or metalloid species such as Li, MgHal, SnR₃, B(OR)₂, SiR₃, GeR₃, and the like. Suitable catalysts for this reaction include bases such as NaH, nBuLi, LDA, LiHMDS, H₂NR, HNR₂, NR₃, nBu₄NF, EtMgHal etc., R in Scheme 10 may be hydrogen, Ac and the like. Typically the reaction is carried out in a suitable solvent such as Et₂O, THF, DME, toluene and the like, and at an appropriate temperature, usually between −100° C. and 25° C. The reaction is allowed to proceed for an appropriate length of time, usually from 15 minutes to 24 hours. The intermediate bearing the —OR group may be isolated and purified as described above, partially purified or carried on to the next step without purification. Elimination of the —OR group to provide the alkene derivative may be accomplished using a variety of methods well known to those skilled in the art. For example, the intermediate may be contacted with POCl₃ in a solvent such as pyridine and stirred at a suitable temperature, typically between 0° C. and 150° C., for an appropriate amount of time, usually between 1 h and 48 h. The product from the reaction can be isolated and purified employing standard techniques, such as solvent extraction, chromatography, crystallization, distillation and the like.

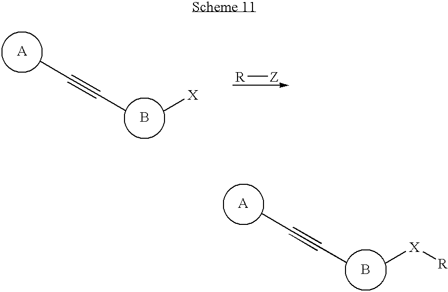

Another embodiment of the present invention is depicted in Scheme 11. An alkynyl-heterocycle (prepared using synthetic chemistry techniques well known in the art) bearing a reactive functional group X is contacted with a species R-Z. In Scheme 11, A and B are as defined above and X is OH, SH, NHR′ and the like. R is a moiety containing at least one isotope such as ¹¹C, ¹³C, ¹⁴C, ¹⁸F, ¹⁵O, ¹³N, ³⁵S, ²H, and ³H and Z is a leaving group such as is halogen, fluorosulfonate, trifluoromethanesulfonate, alkyl- or arylsulfonate and the like. The heterocyclic alkyne is reacted with R-Z in the presence of a suitable catalyst, typically a base such as K₂CO₃, Cs₂CO₃, NaOH, KOH, DBU, DBN, DABCO, NaH, nBuLi, LDA, LiHMDS, H₂NR, HNR₂, NR₃ and the like. The reaction is performed in a suitable solvent such as THF, DME, MeCN, DMF etc. at a temperature of −78° C. up to about 200° C. with from 0° C. to 100° C. being typically preferred. The time for the reaction is from a few minutes up to several hours, with a time range between one minute and one hour typically being sufficient. The product from the reaction is isolated and purified employing standard techniques, usually high-performance liquid chromatography (HPLC) and the like.

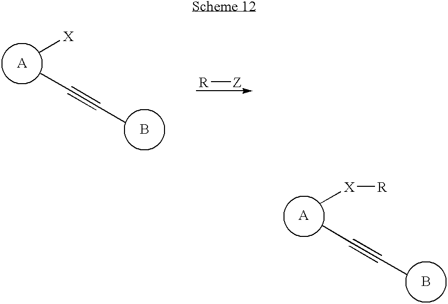

In a further embodiment of the invention illustrated in Scheme 12, a compound similar to the alkyne in Scheme 11 but bearing a reactive group X attached to heterocycle A, is reacted with a species R-Z in a manner analogous that described for the compound in Scheme 11.

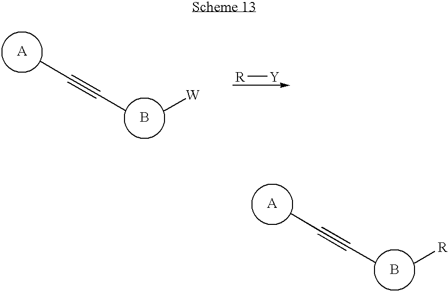

In yet another embodiment of the present invention, depicted in Scheme 13, an alkynyl-heterocycle (prepared using synthetic chemistry techniques well known in the art) bearing a reactive functional group W is reacted with a species R—Y, bearing a reactive functional group Y. In Scheme 13, A and B are as defined above and R is a moiety containing at least one isotope such as ¹¹C, ¹³C, ¹⁴C, ¹⁸F, ¹⁵O, ¹³N, ³⁵S, ²H, and ³H. Y and W are functional groups which are capable of undergoing a transition metal-catalyzed cross-coupling reaction. For example, Y is a group such as halogen, acyloxy, fluorosulfonate, trifluoromethanesulfonate, alkyl- or arylsulfonate, alkyl- or arylsulfinate, alkyl- or arylsulfide, phosphate, phosphinate and the like, and W is hydrogen or a metallic or metalloid species such as Li, MgHal, SnR₃, B(OR)₂, SiR₃, GeR₃, and the like. The coupling may be promoted by a homogeneous catalyst such as Pd₂(dba)₃, PdCl₂(PPh₃)₂, or by a heterogeneous catalyst such as Pd on carbon in a suitable solvent (e.g. THF, DME, MeCN, DMF etc.). Sometimes a co-catalyst such as P(oTol)₃, As(Ph)₃ and the like and a base (e.g. NEt₃, K₂CO₃ etc.) will also be present in the reaction mixture. The coupling reaction typically proceeds at a temperature of −78° C. up to about 200° C. with from 0° C. to 120° C. being typically preferred. The time for the reaction is from a few minutes up to several hours, with a time range between one minute and one hour typically being sufficient. The product from the reaction is isolated and purified employing standard techniques, usually high-performance liquid chromatography (HPLC) and the like.

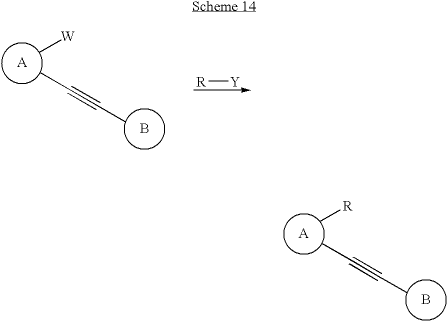

In a further embodiment of the invention illustrated in Scheme 14, a compound similar to the alkyne in Scheme 13 but bearing a reactive group W attached to heterocycle A, is reacted with a species R—Y in a manner analogous that described for the compound in Scheme 13.

Below are examples of specific embodiments for carrying out the present invention. The examples are offered for illustrative purposes only, and are not intended to limit the scope of the present invention in any way.

### Compound 1

### 3-Bromo-5-methoxypyridine

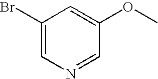

To a solution of NaOMe (1.89 g, 35.0 mmol) in DMF (50 mL) at 60° C. was added 3,5-dibromopyridine (5.14 g, 21.7 mmol). The reaction was stirred for 2.5 h, then cooled to room temperature and stirred for an additional 18 h, quenched with H₂O (ca. 15 mL) and partitioned in a separatory funnel with diethyl ether (100 mL) and H₂O (200 mL). The aqueous layer was washed with 2 additional portions of diethyl ether (2×100 mL). The combined diethyl ether extracts were then back extracted with 50% diluted sat. NaCl (50 mL) then dried over MgSO₄, filtered and concentrated to dryness in vacuo to obtain 3-bromo-5-methoxypyridine as a white solid. ¹H NMR (CDCl₃, 300 MHz) δ 8.30 (d, 1H), 8.25 (d, 1H), 7.37 (dd, 1H), 3.87 (s, 3H).

### Compound 2

### 3-Methoxy-5-[(2-methyl-1,3-thiazol-4yl)ethynyl]pyridine

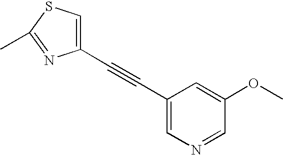

3-Bromo-5-methoxypyridine (392 mg, 2.09 mmol) and 2-methyl-4-[(trimethylsilyl)ethynyl]-1,3-thiazole (339 mg, 1.74 mmol) were added to a deoxygenated, 40° C. DMF (20 mL) solution of triphenylphosphine (73 mg, 0.27 mmol), bis-triphenylphosphine palladium dichloride (98 mg, 0.14 mmol), CuI (53 mg, 0.27 mmol), tetrabutylammonium iodide (257 mg, 0.696 mmol), and triethylamine (879 mg, 1.21 mL, 8.7 mmol). The reaction was warned to 50° C., and tetrabutylammonium fluoride (1.83 mmol, 1.83 mL of a 1.0M solution in THF) was added slowly over 1.5 hours. The reaction was then cooled to ambient temperature and poured into a separatory funnel containing 1:1 hexanes:EtOAc (150 mL) where it was washed with 50% dilute brine (4×50 mL), dried (MgSO₄), filtered, and concentrated in vacuo. The crude residue was chromatographed on silica gel, eluting with 2:1 hexanes:EtOAc to afford 3-methoxy-5-[(2-methyl-1,3-thiazol-4-yl)ethynyl]pyridine as an off-white solid that was then dissolved in diethyl ether (15 mL) and precipitated from solution as the white hydrochloride salt upon treatment with 1M HCl in diethyl ether (5 mL). ¹H NMR (CD₃OD, 300 MHz) δ 8.73 (s, 1H), 8.66 (d, 1H), 8.39 (m, 1H), 7.98 (s, 1H), 4.11 (s, 3H), 2.78 (s, 3H). MS (ESI) 230.9 (M⁺+H).

### Compound 3

### 5-[(2-Methyl-1,3-thiazol-4-yl)ethynyl]pyridin-3-ol

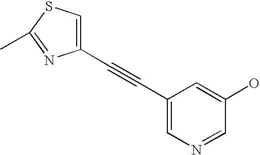

To a solution of toluene (20 mL) and 3-methoxy-5-[(2-methyl-1,3-thiazol-4-yl)ethynyl]pyridine (0.30 g, 1.33 mmol) was added AlBr₃ (8.0 mL in CH₂Cl₂, 8.0 mmol). The solution is stirred at ambient temperature for 3 h, quenched with 10% NaOH, extracted with CH₂Cl₂ (×3), and the aqueous layer neutralized with 10% HCl. The aqueous layer was extracted with CH₂Cl₂ (×3), dried over MgSO₄, filtered and evaporated. The crude material was purified by RPHPLC to yield a white solid. ¹H NMR (CD₃OD, 300 MHz) δ 8.18 (s, 1H), 8.09 (d, 1H), 7.74 (s, 1H), 7.34 (dd, 1H), 3.34 (s, 1H), 2.72 (s, 3H). MS (ESI) 217.1 (M⁺+H).

### Compound 4

### 3-Ethynyl-5-methoxypyridine

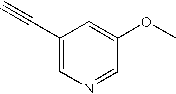

To a degassed solution of triethylamine (20 mL) was added 3-bromo-5-methoxy pyridine (6.3 g, 34 mmol), Pd(PPh₃)₄ (0.4 g, 0.4 mmol), CuI (0.005 g, 0.4 mmol), and TMS acetylene (5.0 g, 51 mmol). The solution was heated to 55° C. for 18 h, cooled to ambient temperature, diluted with diethyl ether and extracted with water (×3). To the organic layer was added TBAF (50 mL (1 M in THF), 50 mmol) and the solution stirred at ambient temperature for 30 minutes. The solution was extracted twice with water, dried over MgSO₄, filtered and evaporated to yield and off white solid. ¹H-NMR (CDCl₃, 500 MHz) δ 8.39 (d, 1H), 8.34 (d, 1H), 7.28 (m, 1H), 3.88 (s, 1H)

### Compound 5

### 4-Bromo-2-methyl-1,3-thiazole d₃

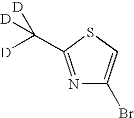

To a solution of 2,4-dibromothiazole (10 g, 41 mmol) in diethyl ether (100 mL) and THF (20 mL) at −78° C., was added nBuLi (46 mmol) over 30 minutes. The solution was stirred at −78° C. for a further 1 h and CuI (3.9 g, 21 mmol) and CD₃I were added along with additional THF (20 mL). The solution was allowed to slowly warm to ambient temperature over 4 h. The reaction was quenched and washed twice with water, dried over Mg SO₄, filtered and evaporated. The crude material is purified by column chromatograph on SiO₂ with 5% EtOAc:hexanes as the eluent to yield a pale yellow oil. ¹H-NMR (CDCl₃, 500 MHz) δ 7.09 (s, 1H)

### EXAMPLE 1A

### 3-Methoxy-5-[(2-methyl-1,3-thiazol-4-yl)ethynyl]pyridine d₃

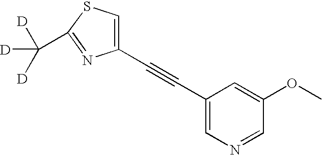

To a degassed solution of triethylamine (20 mL) and DMF (20 mL) was added 4-bromo-2-methyl-1,3-thiazole d₃ (1.5 g, 8.3 mmol), Pd(PPh₃)₄ (0.5 g, 0.4 mmol), CuI (0.016 g, 0.08 mmol), and 3-ethynyl-5-methoxypyridine (1.1 g, 8.3 mmol). The solution was heated to 70° C. for 18 h, cooled to ambient temperature, diluted with diethyl ether and extracted with water (×3), dried over MgSO₄, filtered and evaporated. The crude material was purified by column chromatograph on SiO₂ with 10 to 50% EtOAc/Hexanes as eluent to yield a white solid. ¹H-NMR (CDCl₃, 500 MHz) □ 8.42 (s, 1H), 8.30 (d, 1H), 7.46 (s, 1H), 7.36 (m, 1H). MS (ESI) 234.0 (M⁺+H).

### EXAMPLE 1B

### 5-[(2-Methyl-1,3-thiazol-4-yl)ethynyl]pyridin-3-ol d₃

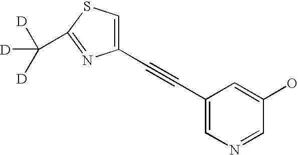

To a solution of CH₂Cl₂ (20 mL) and 3-methoxy-5-[(2-methyl-1,3-thiazol-4-yl)ethynyl]pyridine d₃ (0.10 g, 0.43 mmol) was added AlBr₃ (2.15 mL in CH₂Cl₂, 2.15 mmol). The solution was stirred at ambient temperature for 3 h, quenched with 10% NaOH, extracted with CH₂Cl₂ (×3), and the aqeuous layer neutralized with 10% HCl. The aqueous layer was extracted (×3) with CH₂Cl₂, dried over MgSO₄, filtered and evaporated. The crude material was purified by RPHPLC to yield a white solid. ¹H-NMR (d₃-MeOD, 500 MHz) 8.18 (s, 1H), 8.11 (d, 1H), 7.76 (s, 1H), 7.38 (m, 1H). MS (ESI) 220.1 (M⁺+H).

### Compound 7

### 3-Bromo-5-methylbenzonitrile

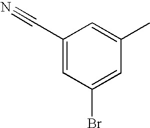

A mixture of 1,3-dibromo-5-methylbenzene (4.97 g, 19.9 mmol), copper (I) cyanide (2.70 g, 30.1 mmol), pyridine (4.85 mL, 60.0 mmol), and N,N-dimethylformamide (35 mL). were heated at 153° C. for 6 h. The reaction was allowed to cool to ambient temperature, poured into a solution of H₂O (200 mL) and NH₄OH (100 mL), and extracted with methyl tert-butyl ether (100 mL×2). The combined organic extracts were washed with saturated aqueous NH₄Cl (200 mL), and the resulting aqueous layer was extracted with methyl tert-butyl ether (50 mL). The combined organic extracts were then washed with saturated aqueous NaHCO₃ (200 mL), and the resulting aqueous layer was extracted with methyl tert-butyl ether (50 mL). The combined organic extracts were dried (MgSO₄), filtered, and concentrated in vacuo. The residue was chromatographed on silica gel with hexanes:EtOAc (99:1→1:99) to afford 3-bromo-5-methylbenzonitrile as an off-white solid. ¹H NMR (CDCl₃, 500 MHz) δ 7.60 (s, 1H), 7.57 (s, 1H), 7.40 (s, 1H), 2.39 (s, 3H).

### Compound 8

### 3-Methyl-5-[(2-methyl-1,3-thiazol-4-yl)ethynyl]benzonitrile

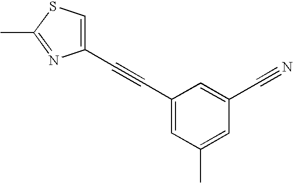

Tetrabutylammonium fluoride (3.2 mL, 1M in THF, 3.2 mmol) was added to a mixture of 3-bromo-5-methylbenzonitrile (394 mg, 2.01 mmol), 2-methyl-4-[(trimethylsilyl)ethynyl]-1,3-thiazole (605 mg, 3.10 mmol), triethylamine (0.60 mL, 4.3 mmol), copper (I) iodide (76 mg, 0.40 mmol), dichlorobis(triphenylphosphine)palladium(II) (138 mg, 0.20 mmol), and N,N-dimethylformamide (4 mL). Nitrogen was bubbled through the resulting mixture for 15 min, and the reaction was heated in a microwave reactor at 100° C. for 15 min. The solvent was removed in vacuo, and the resulting residue was chromatographed on silica gel with hexanes:EtOAc (9:1→1:1) to afford 3-methyl-5-[(2-methyl-1,3-thiazol-4-yl)ethynyl]benzonitrile as an off-white solid. ¹H NMR (CDCl₃, 500 MHz) δ 7.63 (s, 1H), 7.58 (s, 1H), 7.43 (s, 1H), 7.42 (s, 1H), 2.75 (s, 3H), 2.39 (s, 3H). MS (ESI) 239.5 (M⁺+H).

### Compound 9

### 3-Bromo-5-[(2-methyl-1,3-thiazol-4-yl)ethynyl]benzonitrile

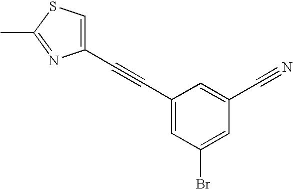

Tetrabutylammonium fluoride (21 mL, 1M in THF, 21 mmol) was added to a mixture of 3,5-dibromobenzonitrile (5.0 g, 19 mmol), 2-methyl-4-[(trimethylsilyl)ethynyl]-1,3-thiazole (3.8 g, 19 mmol), triethylamine (5.5 mL, 40 mmol), copper (I) iodide (730 mg, 3.8 mmol), dichlorobis(triphenylphosphine)palladium(II) (1.4 g, 1.9 mmol), and N,N-dimethylformamide (25 mL). Nitrogen was bubbled through the resulting mixture for 30 min, and the reaction was heated at 90° C. for 5 h. The reaction was allowed to cool to ambient temperature, poured into a solution of H₂O (500 mL) and NH₄OH (150 mL), and extracted with methyl tert-butyl ether (100 mL×3). The combined organic extracts were dried (MgSO₄), filtered, and concentrated in vacuo. The residue was chromatographed on silica gel with hexanes:EtOAc (9:1→1:1) to afford 3-bromo-5-[(2-methyl-1,3-thiazol-4-yl)ethynyl]benzonitrile as a light-brown solid. ¹H NMR (CDCl₃, 500 MHz) δ 7.90 (t, J=1.6 Hz, 1H), 7.76-7.73 (m, 2H), 7.46 (s, 1H), 2.75 (s, 3H). MS (ESI) 303.2 (M⁺+H).

### Compound 10

### 3-[(2-Methyl-1,3-thiazol-4-yl)ethynyl]-5-(trimethylstannyl)benzonitrile

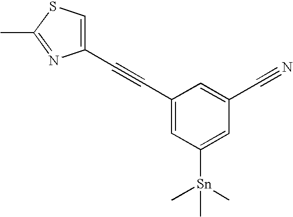

A solution of 3-bromo-5-[(2-methyl-1,3-thiazol-4-yl)ethynyl]benzonitrile (403 mg, 1.33 mmol), hexamethylditin (525 mg, 1.60 mmol), tetrakis(triphenylphosphine)palladium(0) (154 mg, 0.133 mmol), and degassed tetrahydrofuran (4 mL) were heated in a microwave reactor at 110° C. for 1 h. The reaction was poured into H₂O (50 mL) and extracted with methyl tert-butyl ether (50 mL). The organic extract was dried (MgSO₄), filtered, and concentrated in vacuo. The residue was chromatographed on silica gel with hexanes:EtOAc (9:1→3:1) to afford 3-[(2-methyl-1,3-thiazol-4-yl)ethynyl]-5-(trimethylstannyl)benzonitrile as a slightly orange solid. ¹H NMR (CDCl₃, 500 MHz) δ 7.91-7.82 (m, 1H), 7.73 (t, J=1.6 Hz, 1H), 7.73-7.65 (m, 1H), 7.43 (s, 1H), 2.75 (s, 3H), 0.42-0.29 (m, 9H). MS (ESI) 388.9 (M⁺+H).

### Compound 11

### 3-Cyano-5-[(2-methyl-1,3-thiazol-4-yl)ethynyl]phenylboronic acid

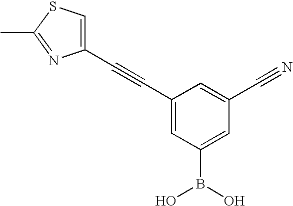

Nitrogen was bubbled through a mixture of 3-bromo-5-[(2-methyl-1,3-thiazol-4-yl)ethynyl]benzonitrile (404 mg, 1.33 mmol), bis(pinacolato)diboron (373 mg, 1.47 mmol), potassium acetate (500 mg, 5.09 mmol), dichloro[1,1′-bis(diphenylphosphino)ferrocene]-palladium(II) dichloromethane adduct (67 mg, 0.082 mmol), and N,N-dimethylacetamide (4 mL) for 1 h. The reaction was heated in a microwave reactor at 110° C. for 20 min, poured into H₂O (50 mL), and extracted with methyl tert-butyl ether (20 mL×4). The combined organic extracts were washed with brine (30 mL), dried (MgSO₄), filtered, and concentrated in vacuo. The residue was purified by reverse-phase HPLC to afford of 3-cyano-5-[(2-methyl-1,3-thiazol-4-yl)ethynyl]phenyl]boronic acid as a white solid. ¹H NMR (CD₃OD, 500 MHz) δ 8.08 (s, 1H), 7.99 (s, 1H), 7.94 (s, 1H), 7.76 (s, 1H), 2.75 (s, 3H). MS (ESI) 268.8 (M⁺+H).

### EXAMPLE 2

### [¹¹C] 3-methoxy-5-[(2-methyl-1,3-thiazol-4-yl)ethynyl]pyridine d₃

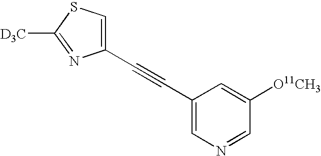

An N-14 gas target containing 1% oxygen was irradiated with an 11 MeV proton beam generating [¹¹C]CO₂. The [¹¹C]CO₂ was trapped at room temperture inside ⅛″ o.d. copper tubing packed with graphite spheres (carbosphere), isolated from the atmosphere by switching a four-port, two-way valve, and set inside a lead container. The [¹¹C]CO₂ was transported to the radiochemistry laboratory. The [¹¹C]CO₂ was converted to [¹¹C]MeI using a GE Medical Systems PETtrace MeI Microlab. The [¹¹C]MeI produced was trapped in a 0° C. mixture of 5-[(2-methyl-1,3-thiazol-4-yl)ethynyl]pyridin-3-ol d₃ (Compound 6, 0.3 mg) in DMF (0.2 mL) containing cesium carbonate. When the amount of radioactivity in this mixture peaked, the mixture was transferred to a vial at 100° C. containing a small amount of cesium carbonate. The reaction mixture was heated for four minutes at 100° C., diluted with H₂O (0.8 mL) and injected onto the HPLC (Waters C18 Xterra, 7.8×150 mm, 15 minute linear gradient, 20% MeCN:(95:5:0.1 H₂O:MeCN:TFA) to 90% MeCN, 3 mL/min). The peak corresponding to [¹¹C] 3-methoxy-5-[(2-methyl-1,3-thiazol-4-yl)ethynyl]pyridine d₃ was collected (˜5 minute retention time), most of the solvent was removed in vacuo, and transferred to a capped vial using physiologic saline as a rinse to give 67 mCi of [¹¹C] 3-methoxy-5-[(2-methyl-1,3-thiazol-4-yl)ethynyl]pyridine d₃. This material coeluted (9 minute retention time) with the authentic standard by HPLC (Waters C18 Symmetry, 4.6×250 mm, 15 minute linear gradient, 20% MeCN:(95:5:0.1 H₂O:MeCN:TFA) to 90% MeCN, 1 mL/min).

### EXAMPLE 3

### [¹¹C] 3-Methoxy-5-[(2-methyl-1,3-thiazol-4-yl)ethynyl]pyridine

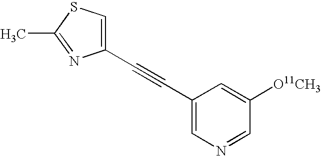

The same procedure used for Example 4 was followed for Example 5 except that 5-[(2-methyl-1,3-thiazol-4-yl)ethynyl]pyridin-3-ol (Compound 2) was used as the precursor.

### EXAMPLE 4

### [¹¹C] 3-Methyl-5-[(2-methyl-1,3-thiazol-4-yl)ethynyl]benzonitrile

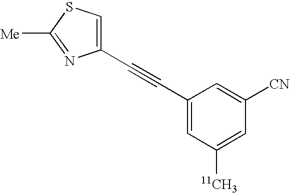

A solution of Pd₂(dba)₃ (˜1 mg) and P(oTol)₃ (˜1.3 mg) in degassed DMF (0.2 mL) was degassed for at least fifteen minutes prior to use. A solution of 3-[(2-methyl-1,3-thiazol-4-yl)ethynyl]-5-(trimethylstannyl)benzonitrile (Compound 10, ˜1.5 mg) in DMF (0.1 mL) was degassed prior to use. [¹¹C]MeI was bubbled into the palladium solution at room temperature and allowed to stand for two minutes. This mixture was transferred to the stannane solution and heated at 120° C. for five minutes. The reaction mixture was diluted with H₂O (0.5 mL) and filtered through a Phenomenex C18-SD Empore Disc Cartridge and injected onto the HPLC (Waters C18 Xterra, 7.8×150 mm, 10 minute linear gradient, 20% MeCN:(95:5:0.1 H₂O:MeCN:TFA) to 90% MeCN, 3 mL/min, hold at 90% MeCN for 10 minutes). The peak corresponding to [¹¹C] 3-methyl-5-[(2-methyl-1,3-thiazol-4-yl)ethynyl]benzonitrile was collected (˜9.5 minute retention time), most of the solvent was removed in vacuo, and was transferred to a capped vial using physiologic saline as a rinse. The [¹¹C]-labeled material coeluted (12 minute retention time) with an authentic sample of Example 3 by HPLC (Waters C18 Symmetry, 4.6×250 mm, 10 minute linear gradient, 20% MeCN:(95:5:0.1 H₂O:MeCN:TFA) to 90% MACN, hold at 90% MeCN for ten minutes, 1 mL/min).

### Compound 12

### (5-Bromopyridin-3-yl)methanol

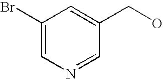

5-Bromonicotinic acid (2.07 g, 10.2 mmol) was suspended in dry DME (15 mL) and cooled in an ice/methanol bath. Triethylamine (1.6 mL, 11.5 mmol) was added to afford a clear solution and isobutyl chloroformate (1.4 ml, 10.8 mmol) was then added to afford a thick suspension. NaBH₄ (788 mg, 20.8 mmol) in H₂O (5 ml) was added dropwise. The cooling bath was removed and the reaction was allowed to warm to ambient temperature overnight. The reaction was quenched by addition of 10% HCl (aqueous), the resulting pale yellow solution was basified by the addition of solid K₂CO₃. The resulting suspension was concentrated in vacuo, the mixture was diluted with EtOAc (200 mL), and washed with water (200 mL), brine (200 mL), dried over Na₂SO₄, and filtered. The filtrate was concentrated in vacuo, and the residue was purified by column chromatography eluting with hexane:EtOAc (3:2) to afford (5-bromopyridin-3-yl)methanol as a clear oil.

### Compound 13

### {5-[(2-Methyl-1,3-thiazol-4-yl)ethynyl]pyridin-3-yl}methanol

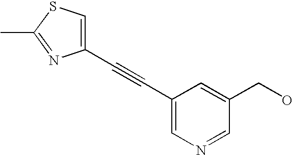

PdCl₂ (29.6 mg, 0.17 mmol) and CuI (95.5 mg, 0.50 mmol) were combined in DME (16 mL), and argon gas was bubbled through the suspension for several minutes before triethylamine (3.4 mL, 24.4 mmol) was added. Argon gas was bubbled through the resulting dark suspension while it was warmed to 70° C. in an oil bath. PPh₃ (181 mg, 0.69 mmol) was added and 2-methyl-4-[(trimethylsilyl)ethynyl]-1,3-thiazole (1.03 g, 5.3 mmol), and (5-bromopyridin-3-yl)methanol (892.1 mg, 4.74 mmol) were added as a solution in DME (7 ml). Tetrabutylammonium fluoride (5.0 mL of 1.0 M solution in tetrahydrofuran, 5.0 mmol) was added over 10 min. Solids appeared in the flask after the addition was completed and the reaction mixture was heated at 70° C. overnight. The reaction mixture was allowed to cool to 25° C. TLC analysis showed no starting (5-bromopyridin-3-yl)methanol present. The reaction mixture was concentrated in vacuo, diluted with EtOAc (300 mL), and filtered. The filtrate was washed with H₂O (100 mL), brine (100 mL), dried over Na₂SO₄, filtered, and concentrated in vacuo to afford a dark oil which partially solidified when under high vacuum. The crude product was purified by column chromatography eluting with CHCl₃, then MeOH:CHCL₃ (1:40) to afford {5-[(2-methyl-1,3-thiazol-4-yl)ethynyl]pyridin-3-yl}methanol mp 98-99° C. ¹H NMR (CD₃OD, 300 MHz) δ 8.59 (s, 1H), 8.51 (s, 1H), 7.94 (s, 1 H), 7.75 (s, 1 H), 4.68 (s, 2 H), 2.72 (s, 3 H). MS (ESI) m/e 230.9 (M+H)⁺.

### Compound 14

### 3-(Methoxymethyl)-5-[(2-methyl-1,3-thiazol-4-yl)ethynyl]pyridine

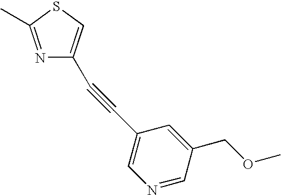

{5-[(2-Methyl-1,3-thiazol-4-yl)ethynyl]pyridin-3-yl}methanol (380 mg, 2.5 mmol) was dissolved in THF (5 mL) under argon and NaH (99 mg, 60% in oil, 2.5 mmol) was added. After 10 min iodomethane (281 mg, 1.98 mmol) was added gradually at 0° C. and allowed to stir overnight. TLC analysis showed no starting material present, and the reaction was quenched by addition of NaHCO₃ (30 mL aqueous), and extracted with EtOAc (20 mL×2). The EtOAc layer was washed with H₂O (20 mL), brine (20 mL), dried over Na₂SO₄, filtered, and concentrated in vacuo. The crude product was purified by column chromatography eluting with hexane to hexane:EtOAc (3:7 to 4:6) to afford 3-(methoxymethyl)-5-[(2-methyl-1,3-thiazol-4-yl)ethynyl]pyridine. ¹H NMR (CD₃OD, 300 MHz) δ 8.71 (s, 1H), 8.52 (s, 1H), 7.84 (s, 1 H), 7.43 (s,1 H), 4.48 (s, 2 H), 3.42(s, 3 H), 2.75 (s, 3 H). MS (ESI) m/e 245.1 (M+H)⁺.

### Compound 15

### 3-methoxy-5-(pyridin-2-ylethynyl)pyridine

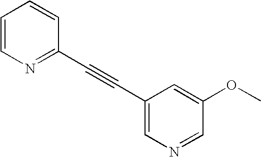

3-Bromo-5-methoxypyridine (Compound 1; 1.00 g, 5.32 mmol) and 2-ethynylpyridine (823 mg, 7.98 mmol) were added to a deoxygenated, 40° C. DMF (35 mL) solution of bis-triphenylphosphine palladium dichloride (300 mg, 0.43 mmol), CuI (162 mg, 0.85 mmol), and triethylamine (2.69 g, 26.6 mmol). The reaction was warmed to 75° C. and stirred under Ar for 6 hours, then cooled to ambient temperature and poured in to a separatory funnel containing 1:1 hexanes:EtOAc (250 mL) where it was washed with 50% dilute brine (4×75 mL), dried (MgSO₄), filtered, and concentrated in vacuo. The crude residue was chromatographed on silica gel, eluting with 1:1 hexanes:EtOAc to afford 3-methoxy-5-(pyridin-2-ylethynyl)pyridine as a tan solid. ¹H NMR (CDCl₃, 300 MHz) δ 8.65 (d, 1H), 8.44 (d, 1H), 8.31 (d, 1H), 7.71 (m, 1H), 7.56 (d, 1H), 7.39 (m, 1H), 7.29 (m, 1H), 3.87 (s, 3H). MS (ESI) 211.1 (M⁺+H).

### Compound 16

### 5-(pyridin-2-ylethynyl)pyridin-3-ol

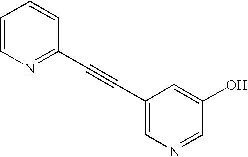

3-methoxy-5-(pyridin-2-ylethynyl)pyridine (Compound 15; 610 mg, 2.90 mmol) was added to an argon-flushed flask containing a stirbar. The flask was then cooled to 0° C. in an ice bath, and to it was added AlBr₃ (20 mL of a 1.0M solution in dibromomethane) with vigorous stirring. The reaction was stirred for 5 min at 0° C., then warmed to ambient temperature, and quenched with saturated sodium potassium tartrate (40 mL). The mixture was poured in to a separatory funnel, diluted with H₂O (200 mL), and washed with DCM (5×75 mL). The organic layers were combined, dried (MgSO₄), filtered, and concentrated in vacuo. The crude residue was chromatographed on silica gel, eluting with 2:1 EtOAc:hexanes to afford 5-(pyridin-2-ylethynyl)pyridin-3-ol as a tan solid. ¹H NMR (CD₃OD, 300 MHz) δ 8.57 (d, 1H), 8.23 (d, 1H), 8.13 (d, 1H), 7.89 (dd, 1H), 7.67 (d, 1H), 7.45 (m, 1H), 7.41 (m, 1H). MS (ESI) 197.1 (M⁺+H).

### EXAMPLE 5

### [¹¹C] 3-Methoxy-5-(Pyridin-2-ylethynyl)pyridine

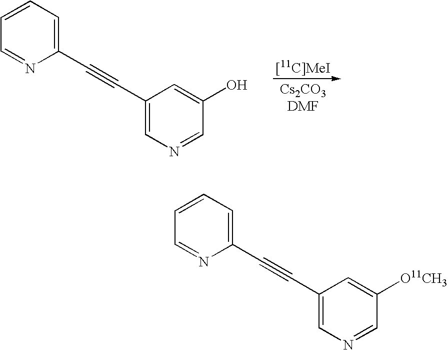

An N-14 gas target containing 1% oxygen was irradiated with an 11 MeV proton beam generating [¹¹C]CO₂. The [¹¹C]CO₂ was trapped at room temperature inside ⅛″ o.d. copper tubing packed with carbosphere, isolated from the atmosphere by switching a four-port, two-way valve, and set inside a lead container. The [¹¹C]CO₂ was transported to the radiochemistry laboratory and was converted to [¹¹C]MeI using a GE Medical Systems PETtrace MeI Microlab. The [¹¹C]MeI produced was trapped in a 0° C. mixture of 5-(pyridin-2-ylethynyl)pyridin-3-ol (Compound 16; 0.3 mg) in DMF (0.2 mL) containing cesium carbonate. When the amount of radioactivity in this mixture peaked, the mixture was transferred to a vial at 100° C. containing a small amount of cesium carbonate. The reaction mixture was heated for four minutes at 100° C., diluted with H₂O (0.8 mL) and injected onto the HPLC (Waters C18 Xterra, 7.8×150 mm, 15 minute linear gradient, 20% MeCN:(95:5:0.1 H₂O:MeCN:TFA) to 90% MeCN, 3 mL/min). The peak corresponding to [¹¹C] 3-methoxy-5-(pyridin-2-ylethynyl)pyridine was collected (˜5 minute retention time), most of the solvent was removed in vacuo, and was transferred to a capped vial using physiologic saline as a rinse to give 50 mCi of [¹¹C] 3-methoxy-5-(pyridin-2-ylethynyl)pyridine

### EXAMPLE 6

### [¹¹F] 3-(Fluoromethoxy)-5-(pyridin-2-ylethynyl)pyridine d₂

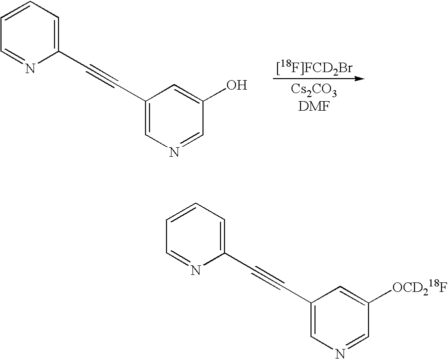

[¹⁸F]F⁻ was produced by 11 MeV proton bombardment of [¹⁸O]H₂O and passing the target contents through an anion exchange resin to recover the [¹⁸O]H₂O. The [¹⁸F]F⁻ was transported to the radiochemistry laboratory on the anion exchange resin which was eluted with 1.5 mL of a mixture of 80% MeCN:20% oxalate* (aq.) solution [*0.05 mL of (200 mg K₂C₂O₄/3 mg K₂CO₃/5 mL H₂O)+0.25 mL H₂O+1.2 mL MeCN]. To the aqueous fluoride solution was added 0.2 mL of 4,7,13,16,21,24-hexaoxa-1,10-diazabicyclo[8.8.8]hexacosane (Kryptofix222; 36 mg/mL MeCN) and the fluoride was dried at 95° C. (oil bath) under vacuum with an argon flow. Additional aliquots of MeCN (3×0.7 mL) were added for azeotropic drying. The oil bath was lowered, and after ˜1 minute, a solution of CD₂Br₂ (0.05 mL) in MeCN (1 mL) was added and the oil bath was raised. An argon stream was used to distill the [¹⁸F]FCD₂Br into a vial at 0° C. containing 5-(pyridin-2-ylethynyl)pyridin-3-ol (Compound 16; 0.5 mg) in DMF (0.2 mL) containing a small amount of Cs₂CO₃. When the amount of radioactivity trapped reached a peak, the vessel was heated at 100° C. for 5 minutes and the DMF was then removed at 100° C. using an argon stream over 5 minutes. The reaction was diluted with ethanol (0.2 mL) and H₂O (0.5 mL) and injected onto the HPLC (Waters C18 μ Bondapak, 7.8×300 mm, 20 minute linear gradient, 10% MeCN:(95:5:0.1 H₂O:MeCN:TFA) to 90% MeCN, 3 mL/min). The peak corresponding to [¹⁸F] 3-(fluoromethoxy)-5-(pyridin-2-ylethynyl)pyridine d₂ was collected (˜12.5 minute retention time), most of the solvent was removed in vacuo, and was transferred to a capped vial using physiologic saline as a rinse to give 5 mCi of [¹⁸F] 3-(fluoromethoxy)-5-(pyridin-2-ylethynyl)pyridine d₂.

### EXAMPLE 7

### [¹⁸F] 3-(Fluoromethoxy)-5-[(2-methyl-1,3-thiazol-4-yl)ethynyl]pyridine d₂

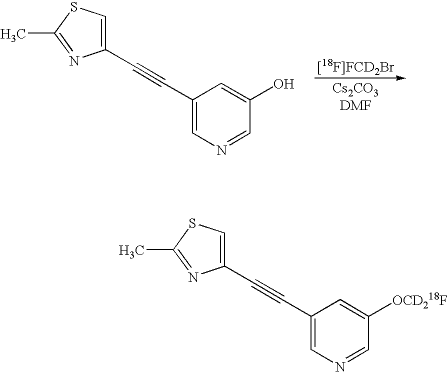

The same procedure was followed for Example 6 except 5-[(2-methyl-1,3-thiazol-4-yl)ethynyl]pyridin-3-ol (Compound 2) was used as the precursor, to give 32 mCi of [¹⁸F] 3-(fluoromethoxy)-5-[(2-methyl-1,3-thiazol-4-yl)ethynyl]pyridine d₂.

### Compound 17

### 6′-Chloro-5-[(2-methyl-1,3-thiazol-4-yl)ethynyl]-3,3′-bipyridine

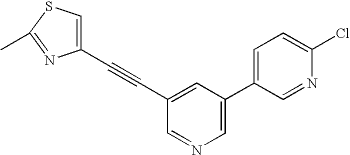

A solution of 6-chloro-3-pyridylboronic acid (1.3 g, 8.3 mmol) in 2:1 DMF:H₂O (30 mL) was degassed for 5 min. 3-Bromo-5-[(2-methyl-1,3-thiazol-4-yl)ethynyl]pyridine (1.5 g, 5.5 mmol), prepared according to the procedure in WO 0116121, K₂CO₃ (1.9 g, 13.8 mmol), Pd(Ph₃P)₄ (320 mg, 0.28 mmol) and n-Bu₄NBr (890 mg, 2.8 mmol) were added. The reaction mixture was heated at 80° C. for 2 h under argon. A further 2 mol % of Pd(Ph₃P)₄ was added and heating continued for 1 h., then the reaction mixture was allowed to cool to 22° C. Water (30 mL) was added and the mixture was extracted with EtOAc (3×100 mL). The combined organic extracts were washed with brine (3×20 mL), dried over MgSO₄, filtered, concentrated under reduced pressure and the residue purified by flash chromatography on silica gel eluting with EtOAc:hexane (1:1) to give 6′-chloro-5-[(2-methyl-1,3-thiazol-4-yl)ethynyl]-3,3′-bipyridine as a solid. MS(ES): 312 (M+H)⁺, ¹H NMR (CDCl₃) δ 8.83 (d, 1 H), 8.76 (d, 1 H), 8.63 (d, 1 H), 8.02 (t, 1 H), 7.86 (dd, 1 H), 7.48 (app. t, 2 H), 2.77 (s, 3 H) ppm.

### EXAMPLE 8

### [¹⁸F] 6′-Fluoro-5-[(2-methyl-1,3-thiazol-4-yl)ethynyl]-3,3′-bipyridine

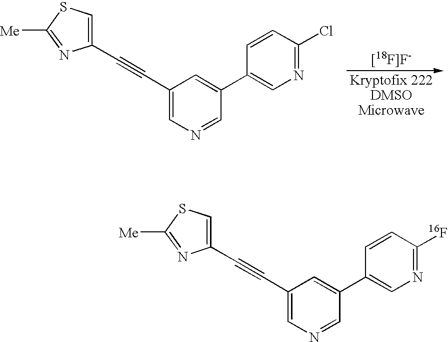

The [¹⁸F]F⁻ containing resin was eluted with 1 mL of a mixture of 80% MeCN:20% oxalate* (aq.) solution [*0.05 mL of (200 mg K₂C₂O₄/3 mg K₂CO₃/5 nL H₂O)+0.25 mL H₂O+1.2 mL MeCN]. A portion of the aqueous fluoride solution (0.5 mL) was transferred to a septum-capped 1 mL v-vial containing a SiC boiling chip in the cavity of the microwave. The microwave settings were coil=high, primary=low and output ˜45 W. This vial had a 1-inch 18 G needle inserted as a vent. To the aqueous fluoride solution was added 0.2 mL of Kryptofix222 (36 mg/mL MeCN) and the fluoride was dried under argon flow using short microwave pulses (5-20 seconds) as needed to remove the water. Additional, aliquots of MeCN (2×0.5 mL) were added to azeotropically dry the fluoride. The vent needle was removed from the vial and a solution of 6′-chloro-5-[(2-methyl-1,3-thiazol-4-yl)ethynyl]-3,3′-bipyridine (Compound 17; 2 mg) in DMSO (0.2 mL) was added and the vial was pulsed with the microwave for 2×15 seconds with a 30 second pause in between. The vial was cooled for ˜1 minute, diluted with H₂O (0.8 mL) and injected onto the HPLC (Waters C18 Xterra, 7.8×150 mm, 15 minute linear gradient, 20% MeCN:(95:5:0.1 H₂O:MeCN:TFA) to 90% MeCN, 3 mL/min). The peak corresponding to [¹⁸F] 6′-fluoro-5-[(2-methyl-1,3-thiazol-4-yl)ethynyl]-3,3′-bipyridine was collected (˜8.5 minute retention time), most of the solvent was removed in vacuo, and was transferred to a capped vial using physiologic saline as a rinse to give 75 mCi of [¹⁸F] 6′-fluoro-5-[(2-methyl-1,3-thiazol-4-yl)ethynyl]-3,3′-bipyridine.

### Compound 18

### 2-Chloro-3-(2-methyl-thiazol-4-ylethynyl)-pyridine

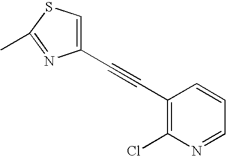

Diisopropylamine (17.6 mmol, 2.5 mL) was dissolved in THF (20 mL) and cooled to −70° C. n-BuLi (2.5 M in hexanes, 17.6 mmol, 7 mL) was added dropwise and the resulting pale yellow solution was stirred at 0° C. for 30 min. The solution was cooled back to −70° C., 2-chloropyridine (17.6 mmol, 2 g) in THF (20 mL) was added dropwise, and the solution stirred at −70° C. for a further 4 h. Iodine (17.6 mmol, 4.5 g) dissolved in THF (15 mL) was added dropwise and stirring was continued for 45 min. at −70° C. The reaction mixture was hydrolyzed with a mixture of H₂O:THF (5:25) at −70° C. followed by addition of H₂O (25 mL) at 0° C. Aqueous sodium thiosulfate and EtOAc were added to the reaction mixture and the 2 layers were separated. The aqueous layer was extracted twice with EtOAc, the organics were combined, dried over Na₂SO₄ and evaporated to dryness to afford a black solid. The crude material was purified by column chromatography on silica gel (20 to 50% CH₂Cl₂ in hexane) to afford a mixture of 2-chloro-3-iodo-pyridine and 2-chloro-3,6-diiodo-pyridine.

A mixture of 2-chloro-3-iodo-pyridine (6:1 mixture with 2-chloro-3,6diiodo-pyridine, 2.1 mmol, 500 mg), 2-methyl-4-trimethylsilanylethynyl-thiazole (3.15 mmol, 614 mg), CuI (0.42 mmol, 80 mg), triethylamine (8.4 mmol, 1.2 mL), PdCl₂(PPh₃)₂ (0.21 mmol, 148 mg), and TBAF (1M in THF, 2.5 mmol, 2.5 mL) in DMF (40 mL) was heated to 65° C. for 3 h. The reaction mixture was cooled to room temperature and EtOAc/brine were added. The 2 layers were separated, the aqueous was extracted twice with EtOAc, the organics were combined, dried over Na₂SO₄ and evaporated to dryness. The crude material was purified by column chromatography on silica gel (10% EtOAc/hexane) to afford 2-chloro-3-(2-methyl-thiazol-4-ylethynyl)-pyridine. ¹H NMR (CDCl₃, 300 MHz) δ 8.35 (dd, 1H), 7.89 (dd, 1H), 7.50 (s, 1H), 7.25 (m, 1H), 2.76 (s, 3H); MS (ESI⁺) 235 (M⁺).

### EXAMPLE 9

### [¹⁸F] 2-Fluoro-3-[(2-methyl-1,3-thiazol-4-yl)ethynyl]pyridine

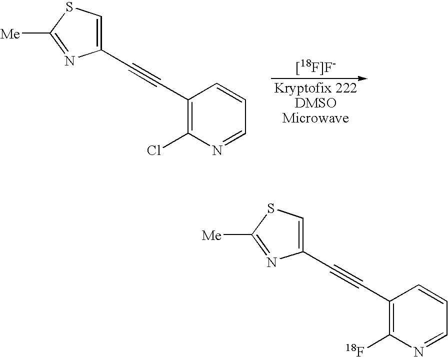

The same procedure was followed for Example 8 except 2-chloro-3-[(2-methyl-1,3-thiazol-4-yl)ethynyl]pyridine (Compound 18) was used as the precursor, to give 36 mCi of [¹⁸F] 2-fluoro-3-[(2-methyl-1,3-thiazol-4-yl)ethynyl]pyridine.

### EXAMPLE 10

### [³H] 3-(Methoxymethyl)-5-[(2-methyl-1,3-thiazol-4-yl)ethynyl]pyridine

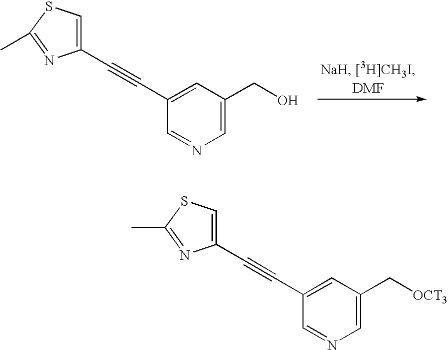

To a 25 mL round bottom flask containing magnetic stir bar, was added sodium hydride (60% suspension in oil, 2.0 mg, excess) under nitrogen atmosphere. Anhydrous n-hexane (2 mL) was added to it and the reaction mixture was stirred at ambient temperature. After stirring for 5 min., organic layer was decanted and anhydrous THF (0.2 mL) was added followed by addition of {5-[(2-methyl-1,3-thiazol-4-yl)ethynyl}pyridin-3-yl}methanol (Compound 13; 0.718 mg, 0.003 mmol) in 0.1 mL anhydrous THF. After stirring for 15 min at room temperature, the reaction mixture was cooled to 0□ C in ice bath and a solution of [³C]methyl iodide (250 mCi, 0.003 mmol) in 0.1 mL toluene (American Radiolabeled Chemicals, Inc.) was added. The cooling bath was removed. After 15 hr., the reaction was quenched by adding ethyl acetate (10 mL) followed by water (5 mL). The aqueous layer was extracted with ethyl acetate (2×5 mL). The combined organic layer was washed with sat. sodium bicarbonate (5 mL), water (5 mL) and brine (5 mL). The organic layer was dried over anhydrous sodium sulfate and evaporated under reduced pressure. The crude product was purified by using semi-preparative HPLC column (Zorbax SB-phenyl, 9.8×250 mm, water containing 0.1% TFA, acetonitrile, 80:20, UV=254 nm, 4 mL/min). The required fractions were collected, combined and acetonitrile was evaporated at reduced pressure. The aqueous solution was passed through Sep-Pak (Waters C-18) cartridge. The cartridge was washed with water followed by eluting with ethanol (10 mL) to yield [³H] 3-(methoxymethyl)-5-[(2-methyl-1,3-thiazol-4-yl)ethynyl]pyridine (26.35 mCi, specific activity=314.8 mCi/mg).

### EXAMPLE 11

### [³H] 3-(Methoxy)-5-(pyridin-2-ylethynyl)pyridine

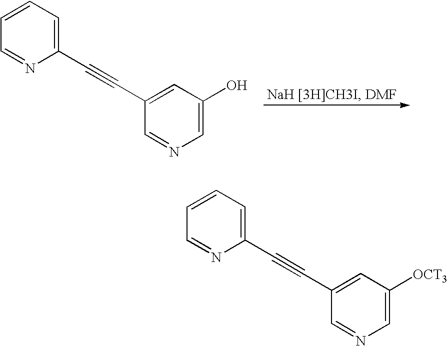

To a 10 mL round bottom flask containing magnetic stir bar, was added sodium hydride (60% suspension in oil, 3.0 mg, excess) under nitrogen atmosphere. Anhydrous n-hexane (2 mL) was added to it and the reaction mixture was stirred at ambient temperature. After 5 min., the organic layer was decanted anhydrous DMF (0.2 mL) was added To this reaction mixture was added 5-(pyridin-2-ylethynyl)pyridin-3-ol (Compound 16; 2.7 mg, 0.013 mmol) in 0.1 mL anhydrous DMF. After stirring for 15 min at room temperature, the reaction mixture was cooled to 0° C. in ice bath and a solution of [³H]methyl iodide (250 mCi, 0.003 mmol) in 0.1 mL toluene (American Radiolabeled Chemicals, Inc.) was added. Cooling bath was removed and reaction mixture was stirred at room temperature. After 15 hr., the reaction was quenched by adding ethyl acetate (10 mL) followed by water (5 mL). The aqueous layer was extracted with ethyl acetate (2×5 mL). The combined organic layer was washed with sat. sodium bicarbonate (5 mL), water (5 mL) and brine (5 mL). The organic layer was dried over anhydrous sodium sulfate and evaporated under reduced pressure. The crude product was purified by using semi-preparative HPLC column (Zorbax SB-phenyl, 9.8×250 mm, water containing 0.1% TFA, acetonitrile, 80:20, UV=254 nm, 4 mL/min). The required fractions were collected, combined and acetonitrile was evaporated at reduced pressure. The aqueous solution was passed through Sep-Pak (Waters C-18) cartridge. The cartridge was washed with water followed by eluting with ethanol (10 mL) to yield [³H]3-methoxy-5-(pyridin-2-ylethynyl]pyridine (33.5 mCi, specific activity=313.2 mCi/mg).

### EXAMPLE 12

### Synthesis of [³H] 3-(methoxy)-5-[(2-methyl-1,3-thiazol-4-yl)ethynyl]pyridine

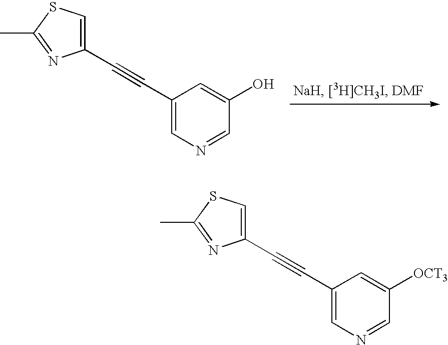

To a 10 mL round bottom flask containing magnetic stir bar, was added sodium hydride (60% suspension in oil, 2.0 mg excess) under nitrogen atmosphere. Anhydrous n-hexane (2 mL) was added to it and the reaction mixture was stirred at ambient temperate. After stirring for 5 min., the organic layer was decanted and anhydrous DMF (0.2 mL) was added. To this reaction mixture was added 5-[(2-methyl-1,3-thiazol-4-yl)ethynyl}pyridin-3-ol (Compound 3; 2.1 mg, 0.01 mmol) in 0.1 mL anhydrous DMF. After stirring for 15 min at room temperature, the reaction mixture was cooled at 0° C. in ice bath and a solution of [³H]methyl iodide (250 mCi, 0.003 mmol) in 0.1 mL toluene (American Radiolabeled Chemicals, Inc.) was added. The cooling bath was removed and reaction mixture was stirred at room temperature. After 15 hr., the reaction was quenched by adding ethyl acetate (10 mL) followed by water (5 mL). The aqueous layer was extracted with ethyl acetate (2×5 mL). The combined organic layer was washed with sat. sodium bicarbonate (5 mL), water (5 mL) and brine (5 mL). The organic layer was dried over anhydrous sodium sulfate and evaporated under reduced pressure. The crude product was purified by using semi-preparative HPLC column (Zorbax SB-phenyl, 9.8×250 mm, water containing 0.1% TFA, acetonitrile, 80:20, UV=254 nm, 4 mL/min). The required fractions were collected, combined and acetonitrile was evaporated at reduced pressure. The aqueous solution was passed through Sep-Pak (Waters C-18) cartridge. The cartridge was washed with water followed by eluting with ethanol (10 mL) to yield [³H3-methoxy-5-(pyridin-2-ylethynyl]pyridine (34. 8 mCi, specific activity 346.2 mCi/mg).

### EXAMPLE 13

### In vitro binding with [³H] 3-(methoxymethyl)-5-[(2-methyl-1,3-thiazol-4-yl)ethynyl]pyridine

Membranes were prepared as described previously (Ransom R W and Stec N. L. J Neurochem. 1988, 51,830-836.) using whole rat brain, or mGlu5^{+/+} or mGlu5^{-/-} whole mouse brain. Binding assays were performed at room temperature as described previously (Schaffhauser H et al. Mol. Pharmacol. 1998, 53,228-233). with slight modifications. Briefly, membranes were thawed and washed once with assay buffer (50 mM HEPES, 2 MM MgCl₂, pH 7.4), followed by centrifugation at 40,000×g for 20 min. The pellet was resuspended in assay buffer and briefly homogenized with a Polytron.

For protein linearity experiments, increasing concentrations of membrane protein were added to 96-well plates in triplicate and binding was initiated by addition of 20 nM [³H] 3-(methoxymethyl)-5-[(2-methyl-1,3-thiazol-4-yl)ethynyl]pyridine. The assay was incubated for 2 h and non-specific binding was determined using 10 μM MPEP. The binding was terminated by rapid filtration through glass-fiber filters (Unifilter-96 GF/B plate, Packard) using a 96-well plate Brandel cell harvester. Following addition of scintillant, the radioactivity was determined by liquid scintillation spectrometry. Protein measurements were performed by BioRad-DC Protein assay using bovine serum albumin as the standard.

Saturation binding experiments were performed in triplicate with increasing concentrations of [³H] 3-(methoxymethyl)-5-[(2-methyl-1,3-thiazol-4-yl)ethynyl]pyridine (1 pM to 100 nM). The time course of association was measured by the addition of 10 nM [³H] 3-(methoxymethyl)-5-[(2-methyl-1,3-thiazol-4-yl)ethynyl]pyridine to the membranes at different time points (0-240 min), followed by filtration. Dissociation was measured by the addition of 100 μM unlabeled methoxymethyl-MTEP at different time points to membranes previously incubated for 3 h with 10 nM [³H] 3-(methoxymethyl)-5-[(2-methyl-1,3-thiazol-4-yl)ethynyl]pyridine. For competition experiments, 100 μg membrane protein and 10 nM [³H] 3-(methoxymethyl)-5-[(2-methyl-1,3-thiazol-4-yl)ethynyl]pyridine was added to wells containing increasing concentration of the test compound in duplicate (3-(methoxymethyl)-5-[(2-methyl-1,3-thiazol-4-yl)ethynyl]pyridine or MPEP).

### Western Blot Analysis of mGlu5 Receptor Protein

Brain hemispheres were homogenized in 20 volumes (w/v) of ice-cold homogenization buffer (PBS/0.1% CHAPS containing a protease inhibitor cocktail (Calbiochem, La Jolla, Calif.)) using a Dounce homogenizer. Homogenates were then incubated on a tube rotator at 4° C. for 30 min, then centrifuged for 10 min at 10,000×g. Supernatants were added 1:1 to 2× sample buffer (Laemmli U. K., [symbol] Nature 1970, 227, 680-685.) and boiled for 2 minutes. Proteins were separated using 4-12% Tris-Glycine PAGE-Gold precast gels (BioWhittaker, Rockland Me.), then transferred onto PVDF membranes (Millipore, Bedford, Mass.). The membranes were blocked in PBS containing 10% not-fat dried milk and probed with the anti-mGlu5 antibody (Upstate Biotechnology, Lake Placid, N.Y.) diluted 1:5000 in PBS containing 0.1% Tween-20. Anti-rabbit IgG-HRP (Amersham, Arlington Heights, Ill.) was used as the secondary antibody and diluted 1:5000 in PBS containing 0.1% Tween-20. The membranes were developed using enhanced chemiluminescence (Amersham, Arlington Heights, Ill.) followed by exposure to Kodak scientific imaging film (Eastman Kodak, Rochester, N.Y.).

[³H] 3-(Methoxymethyl)-5-[(2-methyl-1,3-thiazol-4-yl)ethynyl]pyridine Binding In Vivo Time course of in vivo binding of [³H] 3-(methoxymethyl)-5-[(2-methyl-1,3-thiazol-4-yl)ethynyl]pyridine in rats. Rats were gently restrained in a plastic cone and the tail was warmed briefly to facilitate vessel dilation. [³H] 3-(methoxymethyl)-5-[(2-methyl-1,3-thiazol-4-yl)ethynyl]pyridine (50 μCi/kg; 1 ml/kg injection volume in isotonic saline) was then administered through a lateral tail vein. At the appropriate time, rats were sacrificed and brain tissue was rapidly dissected on a cooled dissecting tray. Hippocampus and cerebellum were immediately weighed and homogenized in 10 volumes of ice-cold buffer (10 mM potassium phosphate, 100 mM KCl, pH 7.4) using a Polytron. Homogenates (400 μl) were then either placed directly into scintillation vials (total radioactivity) or filtered over GF/B membrane filters (Whatman) and washed twice with 5 ml ice-cold homogenization buffer to separate membrane bound from free radioactivity (Atack, J. R., Neuropsychopharmacology 1999, 20, 255-262). Filters and homogenates were then counted for radioactivity using a Beckman counter.

In vivo binding of [³H] 3-(methoxymethyl)-5-[(2-methyl-1,3-thiazol-4-yl)ethynyl]pyridine in mGlu5 deficient mice. In vivo binding in mGlu5 deficient mice (and wild type controls) was performed by administering [³H] 3-(methoxymethyl)-5-[(2-methyl-1,3-thiazol-4-yl)ethynyl]pyridine (50 μCi/kg; 5 ml/kg injection volume in isotonic saline) through a lateral tail vein. Mice were sacrificed 1 min later and forebrain and cerebellum were rapidly dissected, homogenized, and filtered as detailed above.

In vivo receptor occupancy in rats. For studies to determine the in vivo receptor occupancy of unlabeled compounds, rats were dosed ip with unlabeled compound (dissolved in 50% PEG400; 2 ml/kg injection volume). 1 min. prior to sacrifice, [³H] 3-(methoxymethyl)-5-[(2-methyl-1,3-thiazolyl)ethynyl]pyridine was administered (50 μCi/kg) through a lateral tail vein. Animals were then sacrificed and hippocampus was rapidly dissected, homogenized, and filtered as described above.

Data analysis and statistics. In vitro binding curves were fitted using the Prism GraphPad program (Graphpad Software, San Diego, Calif.). Nonlinear regression analysis was used to calculate IC₅₀ values for in vitro displacement studies and to obtain ID₅₀ values for in vivo experiments. Values expressed are the arithmetic means±SEM or the geometric mean (lower, upper standard error). Differences between treatment groups were assessed by analysis of variance followed by either Dunnett's t-test or Student Neuman Keuls test to identify specific group differences.

### EXAMPLE 14

Male SD rats (150-200 g) were euthanized by decapitation and brain regions dissected. The tissue was homogenized (1:10 w/v) in ice-cold assay buffer I (50 mM Tris, pH 7.5, 0.9% NaCl) and the homogenate centrifuged at 17,000 rpm for 10 min at 4° C. to yield a crude P₁ pellet. This pellet was re-suspended in buffer (6 mg original tissue wet weight/ml). Binding of [³H] 3-(methoxy)-5-(pyridin-2-ylethynyl)pyridine (80 Ci/mmol) determined using concentration range 0.01-30 nM and non-specific binding defined with 10 μM MPEP. Subsequent competition studies carried out in rat cortical homogenates using 1 nM radiotracer. Compounds added in a volume of 100 μl to give a final assay volume of 1 ml. Incubations were initiated by adding membranes (3 mg/ml final concentration) and allowed to proceed for 60 min at room temperature (24±2° C.) before being terminated by rapid filtration over GF/B filters pre-soaked in 0.5% polyethyleneimine using 3×10 ml ice cold 0.9% NaCl, pH 7.4. Radioactivity determined using liquid scintillation spectrometry. Protein was assayed by the method of Bradford (Bradford) Anal. Biochem. 1976, 72, 248-254) with bovine serum albumin as the standard. Binding parameters were determined by non-linear least squares regression analysis using Sigmaplot 5.0 (SPSS Inc., USA).

To determine the association rate of [³H] 3-(methoxy)-5-(pyridin-2-ylethynyl)pyridine in rat cortical membranes, 3 nM radiotracer was incubated with 3 mg/ml tissue (final concentration) for varying times prior to filtration (0.5-120 mins). Values for kₒₙ were calculated using the analytic solution to the ligand-receptor association equation using SigmaPlot. To determine the dissociation rate, membranes were incubated with saturating concentrations of radiotracer concentration of 35 nM to equilibrium, then radiotracer dissociation initiated by addition of excess unlabeled MPEP (10 μM). Dissociation rate constants were determined using a mono-exponential function in SigmaPlot.

### Autoradiography

For [³H] 3-(methoxy)-5-(pyridin-2-ylethynyl)pyridine, autoradiography was carried out in cryostat-cut 20 μm coronal sections of rat brain. Sections were air-dried on Frost+slides and stored at −70° C. until used. Sections were incubated for 1 hour at room temperature in Buffer I in the presence of 1 nM radiotracer to obtain total binding values. Non-specific binding was determined using 10 μM MPEP. Following the incubation period, the sections were washed (2×3 minute) in ice-cold assay buffer followed by a 2 second wash in ice-cold de-ionized water. The sections were then dried rapidly under a cool air stream, then juxtaposed to low resolution (TR5025) Fuji phosphor-imaging plates and stored in the dark at room temperature for 5 days (tritium+standards) before scanning on a phosphoimager (BAS5000) and data subsequently analysed with the MCID 4 (BioImaging) software. For [¹⁸F] 3-(fluoromethoxy)-5-(pyridin-2-ylethynyl)pyridine, both rat and rhesus brain sections. were used All methods were identical to the above, but the air dried sections were exposed to high-resolution phosphor-imaging plates for only 20 min to provide adequate exposure. Images were post-processed using Adobe Photoshop for presentation.

### EXAMPLE 15

### [¹¹C]3-Methyl-5-[(2-methyl-1,3-thiazol-4-yl)ethynyl]benzonitrile

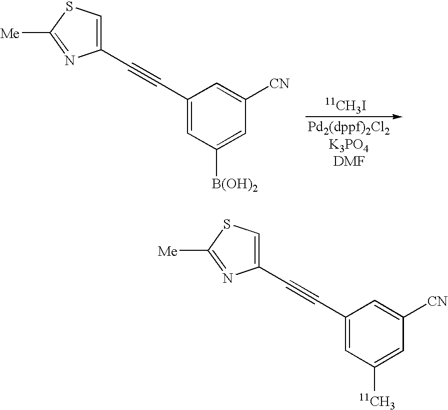

Radionuclides were produced by PETNet Pharmaceuticals, Inc. using a Siemens RDS-111 cyclotron. An N-14 gas target containing 1% oxygen was irradiated-with an 11 MeV proton beam generating [¹¹C]CO₂. The [¹¹C]CO₂ was trapped at room temperature inside ⅛″ o.d. copper tubing packed with carbosphere, isolated from the atmosphere by switching a four-port, two-way valve, and set inside a lead container. The [¹¹C]CO₂ was transported to the radiochemistry laboratory and converted to [¹¹C]MeI using a GE Medical Systems PETtrace MeI Microlab. The [¹¹C]MeI was trapped in a room temperature solution of 1 mg Pd₂(dppf)₂Cl₂ in 0.2 mL of DMF which had been degassed prior to use. After standing for two minutes, this solution was transferred to a solution of 2 mg of 3-cyano-5-[(2-methyl-1,3-thiazol-4-yl)ethynyl]phenylboronic acid in 0.05 mL of degassed DMF and 0.02 mL of 1M K₃PO₄, added just prior to use. This mixture was then added to a 1 mL v-vial positioned in the microwave cavity (Resonance Instruments model 520 microwave, settings: coil=high, primary=low, output ˜45 W). The reaction mixture was pulsed with the microwave for 4×10 second cycles with a 10 second rest in between pulses. After cooling for 30 seconds, the reaction was diluted with 0.5 mL of H₂O, passed through a filter disc and rinsed with 0.1 mL of ethanol. The crude [¹¹C]3-methyl-5-[(2-methyl-1,3-thiazol-4-yl)ethynyl]benzonitrile was purified by preparative HPLC (Waters C18 Xterra, 7.8×150 mm, 10 minute linear gradient, 30% MeCN:(95:5:0.1 H₂O:MeCN:TFA) to 90% MeCN, 3 mL/min). The peak corresponding to [¹¹C]3-methyl-5-[(2-methyl-1,3-thiazol-4-yl)ethynyl]benzonitrile was collected (˜9 minute retention time), most of the solvent was removed in vacuo, and was transferred to a capped vial using physiologic saline as a rinse to give 50 mCi of [¹¹C]3-methyl-5-[(2-methyl-1,3-thiazol-4-yl)ethynyl]benzonitrile.

### EXAMPLE 16

### [¹¹C]3-Methyl-5-[(2-methyl-1,3-thiazol-4-yl)ethynyl]pyridine

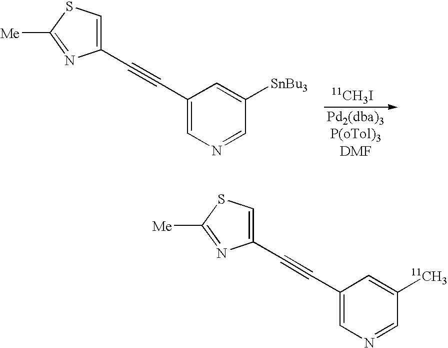

The [¹¹C]MeI was produced as previously described. A mixture of 1 mg of Pd₂(dba)₃ and 1.3 mg of P(oTol)₃ was added together and 0.2 mL of degassed DMF was added and the resulting mixture was degassed using argon gas for at least 10 minutes. The [¹¹C]MeI was trapped at room temperature in this palladium mixture and allowed to stand for two minutes at room temperature. This mixture was then transferred to a solution of 2 mg of 3-tribuylstannyl-5-[(2-methyl-1,3-thiazol-4-yl)ethynyl]pyridine in 0.1 mL of degassed DMF and the reaction mixture was transferred to a 1 mL v-vial in the microwave cavity. A pulse sequence of 5×10 second pulses with 10 seconds in between pulses was used. After cooling for ˜30 seconds, the reaction was diluted with 0.5 mL of H₂O, passed through a filter disc and rinsed with 0.1 mL of ethanol. The crude [¹¹C]3-methyl-5-[(2-methyl-1,3-thiazol-4-yl)ethynyl]pyridine was purified by preparative HPLC (Waters C18 Xterra, 7.8×150 mm, 10 minute linear gradient, 10% MeCN:(95:5:0.1 H₂O:MeCN:TFA) to 90% MeCN, 3 mL/min). The peak corresponding to [¹¹C]3-methyl-5-[(2-methyl-1,3-thiazol-4-yl)ethynyl]pyridine was collected (˜6 minute retention time), most of the solvent was removed in vacuo, and was transferred to a capped vial using physiologic saline as a rinse to give 21 mCi of [¹¹C]3-methyl-5-[(2-methyl-1,3-thiazol-4-yl)ethynyl]pyridine.

### EXAMPLE 17

### [¹⁸F]3-Fluoro-5-[(2-methyl-1,3-thiazol-4-yl)ethynyl]benzonitrile

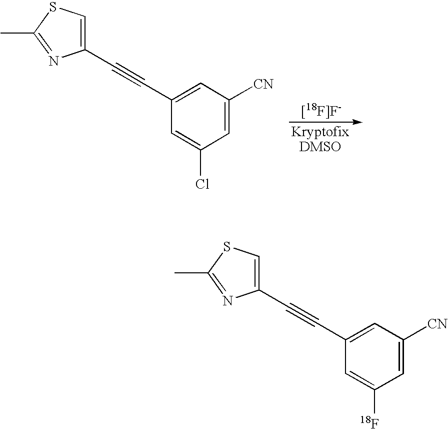

[¹⁸F]F⁻ was produced by 11 MeV proton bombardment of [¹⁸O]H₂O and passing the target contents through an anion exchange resin to recover the [¹⁸O]H₂O. The [¹⁸F]F⁻ was transported to the radiochemistry laboratory on the anion exchange resin which was eluted with 0.5 mL of a mixture of 80% MeCN:20% oxalate* (aq.) solution [*0.05 mL of (200 mg K₂C₂O₄/3 mg K₂CO₃/5 mL H₂O)+0.25 mL H₂O+1.2 mL MeCN] and added to a 1 mL v-vial in the microwave cavity. This vial contained a silcone carbide boiling chip and was vented using a syringe needle. To the aqueous fluoride solution was added 0.2 mL of Kryptofix222 (36 mg/mL MeCN) and the fluoride was dried under argon flow using microwave pulses to heat the aqueous acetonitrile. Additional aliquots of MeCN (2×0.5 mL) were added for azeotropid drying. A solution of 2 mg of 3-chloro-5-[(2-methyl-1,3-thiazol-4-yl)ethynyl]benzonitrile in 0.2 mL of DMSO was added to the microwave vial, the vent needle was removed, and the reaction mixture was pulsed with the microwave for 5×15 seconds with a 30 second pause between pulses. After cooling for one minute, the reaction was diluted with 0.6 mL of H₂O and injected onto the HPLC (Waters C18 μBondapak, 7.8×300 mm, 15 minute linear gradient, 30% MeCN:(95:5:0.1 H₂O:MeCN:TFA) to 90% MeCN, 3 mL/min). The peak corresponding to [¹⁸F]3-fluoro-5-[(2-methyl-1,3-thiazol-4-yl)ethynyl]benzonitrile was collected (˜14 minute retention time), most of the solvent was removed in vacuo, and was transferred to a capped vial using physiologic saline as a rinse to give 7 mCi of [¹⁸F]3-fluoro-5-[(2-methyl-1,3-thiazol-4-yl)ethynyl]benzonitrile.

### EXAMPLE 18

### [¹⁸F]3-Fluoro-5-[(pyridin-2-yl)ethynyl]benzonitrile

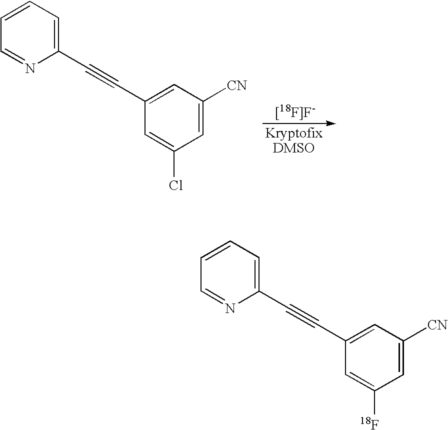

[¹⁸F]3-Fluoro-5-[(pyridin-2-yl)ethynyl]benzonitrile was synthesized using the procedure described above for [¹⁸F]3-fluoro-5-[(2-methyl-1,3-thiazol-4-yl)ethynyl]benzonitrile using 3-chloro-5-[(pyridin-2-yl)ethynyl]benzonitrile as the precursor. HPLC purification (Waters C18 μBondapak, 7.8×300 mm, 15 minute linear gradient, 10% MeCN:(95:5:0.1 H₂O:MeCN:TFA) to 90% MeCN, 3 mL/min, retention time ! 14 minutes) gave 14 mCi of [¹⁸F]3-fluoro-5-[(pyridin-2-yl)ethynyl]benzonitrile.

### EXAMPLE 19

### [¹⁸F]3-(2-Fluoroethoxy)-5-[(2-methyl-d₃-1,3-thiazol-4-yl)ethynyl]pyridine

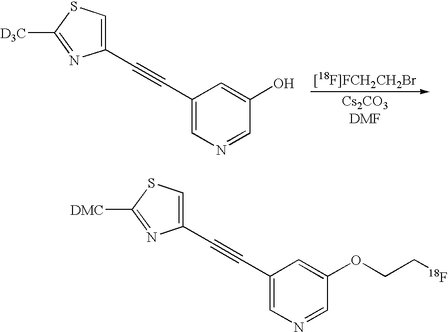

[¹⁸F]F⁻ was produced by 11 MeV proton bombardment of [¹⁸O]H₂O and passing the target contents through an anion exchange resin to recover the [¹⁸O]H₂O. The [¹⁸F]F⁻ was transported to the radiochemistry laboratory on the anion exchange resin which was eluted with 1.5 mL of a mixture of 80% MeCN:20% oxalate* (aq.) solution [*0.05 mL of (200 mg K₂C₂O₄/3 mg K₂CO₃/5 ML H₂O)+0.25 mL H₂O+1.2 mL MeCN]. To the aqueous fluoride solution was added 0.2 mL of Kryptofix222 (36 mg/mL MeCN) and the fluoride was dried at 115° C. (oil bath) under vacuum and argon flow (˜10 mL/min). Additional aliquots of MeCN (3×0.7 mL) were added for azeotropic drying at 115° C. The oil bath was lowered, and after ˜1 minute, a solution of bromoethyltriflate (0.005 mL, Chi et al, JOC, 1987, 52, 658-664) in 1,2-dichlorobenzene (0.7 mL) was added and the oil bath was raised. An argon stream was used to distill the [¹⁸F]FCH₂CH₂Br into a vial at RT containing 3-hydroxy-5-[(2-methyl-d₃-1,3-thiazol-4-yl)ethynyl]pyridine

(0.3 mg) in DMF (0.2 mL) containing a small amount (˜1-2 mg) of Cs₂CO₃. When the amount of radioactivity trapped reached a peak, the mixture was transferred to a 2 mL vial at 100° C. containing a small amount of cesium carbonate. The reaction mixture was heated for five minutes at 100° C., diluted with H₂O (0.8 mL) and purified by HPLC (Waters C18 Xterra, 7.8×150 mm, 15 minute linear gradient, 20% MeCN:(95:5:0.1 H₂O:MeCN:TFA) to 90% MeCN, 3 mL/min). The peak corresponding to [¹⁸F]3-(2-fluoroethoxy)-5-[(2-methyl-d₃-1,3-thiazol-4-yl)ethynyl]pyridine was collected (˜7 minute retention time) in a 50 mL round bottom flask on a rotary evaporator, most of the solvent was removed in vacuo, and was transferred to a capped vial using physiologic saline as a rinse to give 28 mCi of the product.

Scheme 15 relates to Examples 20 through 29:

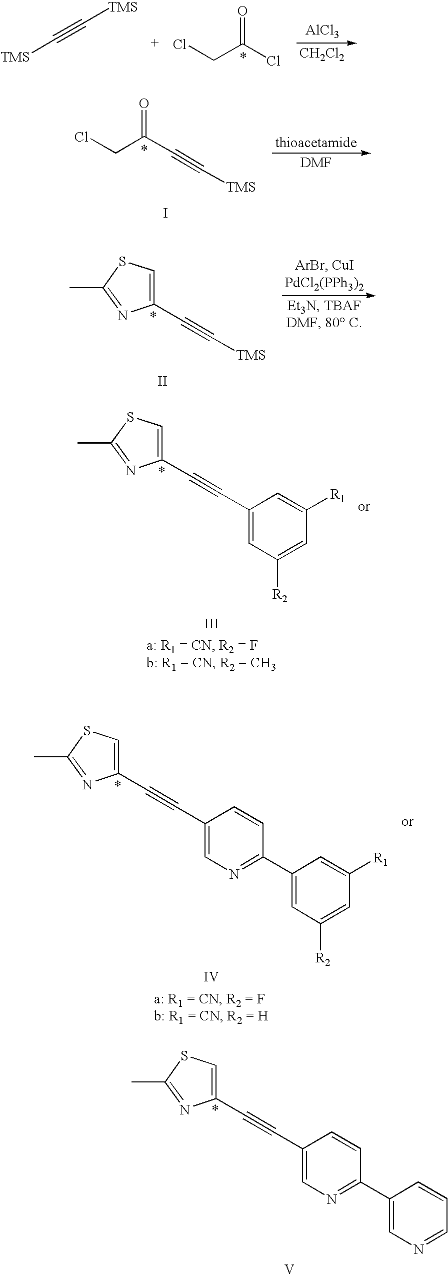

### EXAMPLE 20

### [Carbonyl-¹⁴C]1-chloro-4-(trimethylsilyl)but-3-yn-2-one (1)

A suspension of 325 mg of aluminum chloride in 5 mL of methylene chloride was first stirred at room temperature for twenty minutes and then at 0° C. In a separate flask, dissolved 297 mg of bis(trimethylsilyl)acetylene in 2 mL of methylene chloride and added to 100 mCi of [carbonyl-¹⁴C]chloroacetyl chloride via syringe. This solution was then added dropwise to the aluminum chloride suspension at 0° C. The resulting dark brown mixture was stirred for 1 hr at 0° C., followed by 1 hr at room temperature. It was then cooled back down to 0° C. and quenched with the slow addition of 1 N HCl. The layers were separated and the aqueous portion extracted with methylene chloride (2×5 mL). The organic extracts were then combined and washed with water (1×5 mL) and saturated sodium bicarbonate (1×5 mL). The solution was dried over sodium sulfate, filtered, and concentrated in vacuo at 400 mBar, to afford 91 mCi of a dark brown oil which had a radiochemical purity of 95% by BPLC (Zorbax SB C18 column, 4.6×150 mm, 20% acetonitrile:H₂O (0.1% TFA) to 100% acetonitrile, 15 min linear gradient, 1 mL/min, t_{R}=12.1 min).

### EXAMPLE 21

### [Thiazole-4-¹⁴C]2-methyl-4-[(trimethylsilyl)ethynyl]-1,3-thiazole (II)

A solution of 91 mCi (309 mg) of [carbonyl-¹⁴C]1-chloro-4-(trimethylsilyl)but-3-yn-2-one in 4 mL of dimethylformamide at 0° C. was treated with 171 mg of thioacetamide, after which it was warmed to room temperature and stirred overnight. The reaction mixture was diluted with 5 mL of 1:1 ethyl acetate:hexane and washed with 1:1 H₂O:brine (3×7 mL). The aqueous washings were combined and extracted with 1:1 ethyl acetate:hexane (3×5 mL). The organic extracts were then combined, dried over sodium sulfate, filtered, and concentrated in vacuo at 150 mBar. After flash chromatography on silica gel (2.5% ethyl acetate:hexane eluent) 53 mCi (201 mg) of a yellow oil was obtained which had a radiochemical purity of 89% by HPLC (Zorbax SB C18 column, 4.6×150 mm, 20% acetonitrile:H₂O (0.1% TFA) to 100% acetonitrile, 15 min linear gradient, 1 mL/min, t_{R}=12.4 min).

### EXAMPLE 22

### [Thiazole-4-¹⁴C]3-fluoro-5-[(2-methyl-1,3-thiazol-4-yl)ethynyl]benzonitrile (IIIa)

A solution of 15 mg of 3-bromo-5-fluorobenzonitrile, 5.4 mg of dichlorobis(triphenylphosphine)palladium(II), 3 mg of copper (I) iodide, 54 μL of triethylamine, 4 mCi (15 mg) of [thiazole-4-¹⁴C]2-methyl-4-[(trimethylsilyl)ethynyl]-1,3-thiazole and 1 mL of dimethylformamide was heated to 50° C. under a nitrogen atmosphere. This was followed by the addition of 77 μL of a 1 M solution of tetrabutylammonium fluoride in tetrahydrofuran and the temperature was raised to 80° C. After 3 hr, the reaction mixture was cooled to room temperature, diluted with 5 ml of 1:1 ethyl acetate:hexane and washed with 1:1 brine:H₂O (1×10 mL). The aqueous portion was then extracted with 1:1 ethyl acetate:hexane (2×5 mL). The organic extracts were combined, dried over sodium sulfate, filtered, and concentrated in vacuo. The residue was then dissolved in 3 mL of acetonitrile and filtered through a 0.2 μM filter. After preparative HPLC chromatography (Zorbax XDB C18 250×21.2 mm column, 20% acetonitrile:H₂O (0.1% TFA) to 100% acetonitrile, 60 min linear gradient, 20 mL/min, 2×1.5 mL injections) 1.2 mCi (5.6 mg) of product was obtained which had a radiochemical purity of >99.5% by HPLC (Zorbax SB C18 column, 4.6×150 mm, 40% acetonitrile:H₂O (0.1% TFA) for 20 min, to 100% acetonitrile in 10 min, hold at 100% for 15 min, 1 mL/min, t_{R}=19.3 min) and coeluted with an authentic sample of [Thiazole-4-¹⁴C]3-fluoro-5-[(2-methyl-1,3-thiazol-4-yl)ethynyl]benzonitrile. LC/MS m/z=245.

### EXAMPLE 23

### [Thiazole-4-¹⁴C]3-methyl-5-[(2-methyl-1,3-thiazol-4-yl)ethynyl]benzonitrile (IIIb)

Example 22 was followed, using 3-bromo-5-methylbenzonitrile as the aryl bromide. 800 μCi (3.7 mg) of product was obtained which had a radiochemical purity of >99.5% by HPLC (Zorbax SB C18 column, 4.6×150 mm, 45% acetonitrile:H₂O (0.1% TFA) for 20 min, to 100% acetonitrile in 10 min, hold at 100% for 15 min, 1 mL/min, t_{R}=15.6 min) and coeluted with an authentic sample of [Thiazole-4-¹⁴C]3-methyl-5-[(2-methyl-1,3-thiazol-4-yl)ethynyl]benzonitrile. LC/MS m/z=241.

### EXAMPLE 24

### [Thiazole-4-¹⁴C]3-fluoro-5-[5-([2-methyl-1,3-thiazol-4-yl]ethynyl)pyridin-2-yl]benzonitrile (IVa)

Example 22 was followed, using 3-(5-bromopyridin-2-yl)-5-fluorobenzonitrile as the aryl bromide. 1.1 mCi (6.8 mg) of product was obtained which had a radiochemical purity of >99.5% by HPLC (Zorbax SB C18 column, 4.6×150 mm, 50% acetonitrile:H₂O (0.1% TFA) for 20 min, to 100% acetonitrile in 10 min, hold at 100% for 15 min, 1 mL/min, t_{R}=17.5 min) and coeluted with an authentic sample of [Thiazole-4-¹⁴C]3-fluoro-5-[5-([2-methyl-1,3-thiazol-4-yl]ethynyl)pyridin-2-yl]benzonitrile. LC/MS m/z=322

### EXAMPLE 25

### [Thiazole-4-¹⁴C]3-(5-[(2-methyl-1,3-thiazol-4-yl)ethynyl]pyridin-2-yl)benzonitrile (IVb)

Example 22 was followed, using 3-(5-bromopyridin-2-yl)benzonitrile as the aryl bromide. 1.1 mCi (6.4 mg) of product was obtained which had a radiochemical purity of >99.5% by HPLC (Zorbax SB C18 column, 4.6×150 mm, 45% acetonitrile:H₂O (0.1% TFA) for 20 min, to 100% acetonitrile in 10 min, hold at 100% for 15 min, 1 mL/imin, t_{R}=18.1 min) and co-eluted with an authentic sample of [Thiazole-4-¹⁴C]3-(5-[(2-methyl-1,3-thiazol-4yl)ethynyl]pyridin-2-yl)benzonitrile. LC/MS m/z=304.

### EXAMPLE 26

### [Thiazole-4-¹⁴C]5-(2-methyl-thiazol-4-ylethynyl)-[2,3′]bipyridyl (V)

Example 22 was followed, using 5-bromo-[2,3′]bipyridyl as the aryl bromide. 1.3 mCi (13.2 mg) of product was obtained which had a radiochemical purity of 99.1% by HPLC (Zorbax Rx C8 column, 4.6×250 mm, 40% acetonitrile:H₂O (0.1% TFA) to 100% acetonitrile in 30 min, hold at 100% for 10 min, 1 mL/min, t_{R}=23.6 min) and co-eluted with an authentic sample of [Thiazole-4-¹⁴C]5-(2-methyl-thiazol-4-ylethynyl)-[2,3′]bipyridyl. LC/MS mlz=278.

### EXAMPLE 27

### [U-¹⁴C-phenyl](2-methyl-1,3-thiazol-4-yl)ethynylbenzene

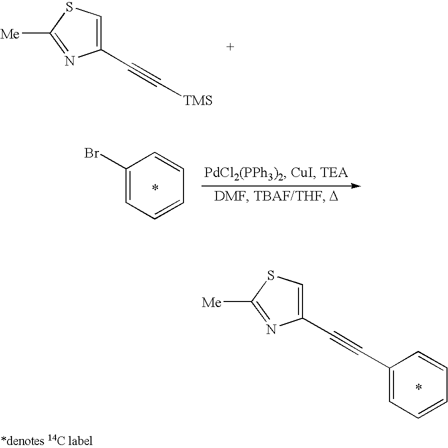

To a solution of [thiazole-4-¹⁴C]2-methyl-4-[(trimethylsilyl)ethynyl]-1,3-thiazole (25 mCi, 123 mCi/mmol, 0.20 mmol) in 1.6 mL of DMF was added [U-¹⁴C]-bromobenzene (45 mg, 0.24 mmol), PdCl₂(PPh₃)₂ (12 mg), CuI (8 mg), and triethylamine (120 μL). The reaction mixture was degassed by bubbling N₂ through for 5 min and heated to 70° C. in an oil bath. A solution of TBAF/THF (120 μL, 1.0 M) was added slowly, and the resulting mixture was aged at 70° C. for 16 h. After the reaction mixture was cooled to room temperature, it was extracted with hexanes/EtOAc (1:1) and brine. The combined organic layers were dried over Na₂SO₄ (anh.) and filtered to give 17.7 mCi of [U-¹⁴C-phenyl](2-methyl-1,3-thiazol-4-yl)ethynylbenzene (HPLC analysis: Zorbax SB-Phenyl, 4.6×250 mm, 45:55:0.1 MeCN:H₂O:TFA, 1.0 mL/min, 30° C., t_{R}=14.82 min, 55.8% radiochemical purity). The crude [U-¹⁴C-phenyl](2-methyl-1,3-thiazol-4-yl)ethynylbenzene was purified by sequential prep-HPLC columns (1. Zorbax SB-Phenyl, 21.2×250 mm, 20 mL/min, gradient: 37% to 42% MeCN in H₂O (0.1% TFA) over 60 min; 2. Waters XTerra RP-18 7 μm, 19×300 mm, 20 mL/min, isocratic: 40:60:0.1 MeCN:H₂O:TFA) to give 4.54 mCi of [U-¹⁴C-phenyl](2-methyl-1,3-thiazol-4-yl)ethynylbenzene, which was diluted with 19.7 mg of unlabeled (2-methyl-1,3-thiazol-4-yl)ethynylbenzene. (HPLC analysis: Zorbax SB-Phenyl, 4.6×250 mm, 45:55:0.1 MeCN:H₂O:TFA, 1.0 mL/min, 30° C., t_{R}=14.93 min, 100% radiochemical purity, 98.3% UV purity at 254 nm).

### EXAMPLE 28

### 3-[³H₃C]-5-[(2-methyl-1,3-thiazol-4-yl)ethynyl]benzonitrile

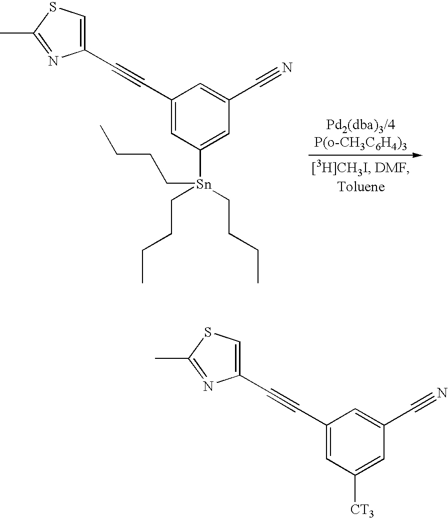

In a dry reaction vial (2 mL), tris(dibenzylideneacetone)dipalladium(0) (4.6 mg, 0.005 mmol) and tri-o-tolylphosphine (6.0 mg, 0.02 mmol) were placed under nitrogen. After addition of DMF (50 uL), the reaction mixture was stirred for 5 min at room temperature and then a solution of [³H]methyl iodide in toluene (200 uL, 100 mCi, SA=80 Ci/mmol, 0.0012 mmol, obtained from American Radiolabeled Chemicals Inc., Saint Louis, Mo. USA) was added. The mixture was further stirred for 3 min at room temperature followed by successive addition of a solution of tributyltin derivative 3-[(2-methyl-1,3-thiazol-4-yl)ethynyl]-5-(tributylstannyl)benzonitrile (3.9 mg, 0.008 mmol) in 200 uL DMF. The resulting mixture was stirred under nitrogen at 50° C. for 15 hr. The crude reaction mixture was concentrated under reduced pressure and purified by semi-preparative HPLC column (Zorbax SB phenyl, water containing 0.1% TFA, acetonitrile, 60:40, 4 mL/min, UV=254 nm, Rf=20 min). The combined HPLC fractions were passed through Waters Sep-Pak^{R} cartridge (Plus C18) and was washed with water and then the product was eluted with ethanol (10 mL) to give 3-[³H₃C]-5-[(2-methyl-1,3-thiazol-4-yl)ethynyl]benzonitrile (3.8 mCi) with specific activity 76.1 Ci/mmol as determined by LC/MS (Agilent MSD-1100 with electrospray ionization). Radiochemical purity of 3-[³H₃C]-5-[(2-methyl-1,3-thiazol-4-yl)ethynyl]benzonitrile was >98% as determined by HPLC (Zorbax SB phenyl, water containing 0.1% TFA:acetonitrile, 60:40, 1 mL/min, UV=254 nm, Rf=20.2 min).

### EXAMPLE 29

### [Thiazole-4-¹⁴C]-3-[(2-Methyl-1,3-thiazol-4-yl)ethynyl]pyridine

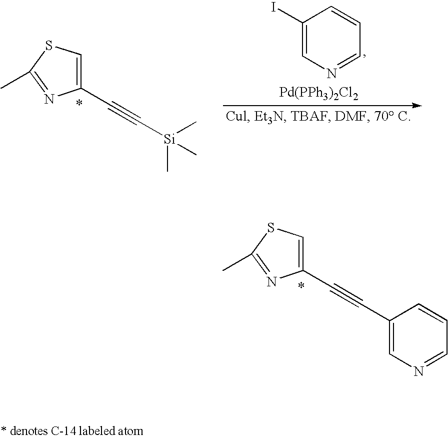

A solution of Pd(II)(PPh₃)₂Cl₂ (3.9 mg, 0.0055 mmol), CuI (2.1 mg, 0.011 mmol) and triethyl amine (77 μL, 0.55 mmol) in 1.5 mL anhydrous DMF was bubbled N₂ through for 2 min. and heated at 40° C. for 10 min. before the addition of 3-iodiopyridine (114 mg, 0.55 mmol). Then the temperature was raised to 70° C. followed by addition of [thiazole-4-¹⁴C]2-methyl-4-[(trimethylsilyl)ethynyl]-1,3-thiazole (7.0 mCi, S.A.=52 mCi/mmol, 0.14 mmol) and a slow addition of TBAF (1.0M in THF, 158 uL, 0.15 mmol) which was effected by a syringe pump. The reaction was stirred at 70° C. overnight. After the reaction was cooled down to room temperature, it was diluted with 20 mL of 1:1 EtOAc/Hexane, then washed with 10 mL of water. The aqueous layer was extracted with 2×10 mL 1:1 EtOAc/Hexane and the combined organic layers were washed with 3×10 mL water and 10 mL brine, then dried over Na₂SO₄ and filtered to give 6.3 mCi crude [thazole-4-¹⁴C-3-[(2-Methyl-1,3-thiazol-4-yl)ethynyl]pyridine (HPLC analysis: Zorbax SB-C8 column, 4.6×250 mm, 15% MeCN-0.1% aq. TFA, 1.0 mL/min., 25° C., t_{R}=18.7 min., 81.8% radiochemical purity) in 85.9% yield. Final purification was effected by automated prep. HPLC separations (Zorbax Rx-C8 column, 21.2×250 mm, 10% MeCN-0.1% aq. TFA, 20 mL/min., 25° C., 7 runs of 0.9 mCi/run) to give 5.4 mCi of [thiazole-4-¹⁴C]-3-[(2-Methyl-1,3-thiazol-4-yl)ethynyl]pyridine with 99.7% radiochemical purity (BPLC analysis: Zorbax SB-C8 column, 4.6×250 mm, 15% MeCN-0.1% aq. TFA, 1.0 mL/min., 25° C., t_{R}=18.9 min.) and a specific activity of 52.4 mCi/mmol.

While the invention has been described: and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A compound represented by Formula I: 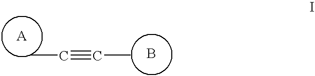
or a pharmaceutically acceptable salt thereof, wherein:
A is a heterocycle optionally substituted with one to five independent halogen, —CN, NO₂, —C₁₋₆alkyl, —C₁₋₆alkenyl, —C₁₋₆alkynyl, —OR¹, —NR¹R², —C(═NR¹)NR²R³, —N(═NR¹)NR²R³, —NR¹COR², —NR¹CO₂R², —NR¹SO₂R⁴, —NR¹CONR²R³, —SR⁴, —SOR⁴, —SO₂R⁴, —SO₂NR¹R², —COR¹, —CO₂R¹, —CONR¹R², —C(═NR¹)R², or —C(═NOR¹)R² substituents; wherein the alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R¹, R², and R³ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁴ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
B is aryl, heterocycle, —C₃₋₂₀cycloalkyl, —C₃₋₂₀cycloalkenyl, —C₃₋₂₀cycloalkadienyl, —C₃₋₂₀cycloalkatrienyl, —C₃₋₂₀cycloalkynyl, —C₃₋₂₀cycloalkadiynyl; optionally substituted with one to five independent halogen, —CN, NO₂, —C₁₋₆alkyl, —C₁₋₆alkenyl, —C₁₋₆alkynyl, —OR⁵, —NR⁵R⁶, —C(═NR⁵)NR⁶R⁷, —N(═NR⁵)NR⁶R⁷, —NR⁵COR⁶, —NR⁵CO₂R⁶, —NR⁵SO₂R⁸, —NR⁵CONR⁶R⁷, —SR⁸, —SOR⁸, —SO₂R⁸, —SO₂NR⁵R⁶, —COR⁵, —CO₂R⁵, —CONR⁵R⁶, —C(═NR⁵)R⁶, —C(═NOR⁵)R⁶, aryl or heterocycle substituents; wherein the allyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁵, R⁶, and R⁷ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁸ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
wherein the compound is isotopically labeled with at least one ¹¹C, ¹³C, ¹⁴C, ¹⁸F, ¹⁵O, ¹³N, ³⁵S, ²H, or ³H atom;
except when A=6-methyl-2-pyridyl then B cannot be 3-methoxyphenyl or unsubstituted phenyl.

2. A compound represented by Formula II: 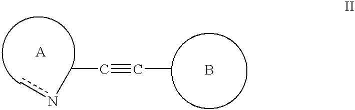
or a pharmaceutically acceptable salt thereof, wherein:
A is pyridinyl, pyrrolyl, imidazolyl, pyridazinyl, pyrimidinyl, pyrazoyl, pyrazinyl, triazolyl, triazinyl, tetrazolyl, tetrazinyl, tetrazepinyl, isoxazolyl, oxazolyl, oxadiazolyl, oxatriazolyl, oxazinyl, oxadiazinyl, isothiazolyl, thiazolyl, thiadazinyl, thiadiazolyl, thiadiazepinyl, dioxazolyl, oxathiazolyl, oxathiazinyl, oxazepinyl, oxadiazepinyl, azepinyl, and diazepinyl, optionally substituted with one to five independent halogen, —CN, NO₂, —C₁₋₆alkyl, —C₁₋₆alkenyl, —C₁₋₆alkynyl, —OR¹, —NR¹R², —C(═NR¹)NR²R³, —N(═NR¹)NR²R³, —NR¹COR², —NR¹CO₂R², —NR¹SO₂R⁴, —NR¹CONR²R³, —SR⁴, —SOR⁴, —SO₂R⁴, —SO₂NR¹R², —COR¹, —CO₂R¹, —CONR¹R², —C(═NR¹)R², or —C(═NOR¹)R² substituents; wherein the alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R¹, R², and R³ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁴ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
B is phenyl, —C₃₋₂₀cycloalkyl, —C₃₋₂₀cycloalkenyl, —C₃₋₂₀cycloalkadienyl, —C₃₋₂₀cycloalkatrienyl, —C₃₋₂₀cycloalkynyl, —C₃₋₂₀cycloalkadiynyl, indenyl, dihydroindenyl, naphthalenyl, dihydronaphthalenyl, pyridinyl, thiazolyl, furyl, dihydropyranyl, dihydrothiopyranyl, piperidinyl, isoxazolyl, pyridazinyl, pyrimidinyl, pyrazinyl, indolyl, quinolinyl, isoquinolinyl, optionally substituted with one to five independent halogen, —CN, NO₂, —C₁₋₆alkyl, —C₁₋₆alkenyl, —C₁₋₆alkynyl, —OR⁵, —NR⁵R⁶, —C(═NR⁵)NR⁶R⁷, —N(═NR⁵)NR⁶R⁷, —NR⁵COR⁶, —NR⁵CO₂R⁶, —NR⁵SO₂R⁸, —NR⁵CONR⁶R⁷, —SR⁸, —SOR⁸, —SO₂R⁸, —SO₂NR⁵R⁶, —COR⁵, —CO₂R⁵, —CONR⁵R⁶, —C(═NR⁵)R⁶, —C(═NOR⁵)R⁶, aryl or heterocycle substituents; wherein the alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁵, R⁶, and R⁷ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁸ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents; and
wherein the compound is isotopically labeled with at least one ¹¹C, ¹³C, ¹⁴C, ¹⁸F, ¹⁵O, ¹³N, ³⁵S, ²H, or ³H atom;
and except when A=6-methyl-2-pyridyl then B cannot be 3-methoxyphenyl or unsubstituted phenyl.

3. The compound of claim 1 wherein A is pyridinyl, pyrrolyl, imidazolyl, pyridazinyl, pyrimidinyl, pyrazoyl, pyrazinyl, triazolyl, triazinyl, tetrazolyl, tetrazinyl, tetrazepinyl, isoxazolyl, oxazolyl, oxadiazolyl, oxatriazolyl, oxazinyl, oxadiazinyl, isothiazolyl, thiazolyl, thiadazinyl, thiadiazolyl, thiadiazepinyl, dioxazolyl, oxathiazolyl, oxathiazinyl, oxazepinyl, oxadiazepinyl, azepinyl, and diazepinyl, optionally substituted with one to five independent halogen, —CN, NO₂, —C₁₋₆alkyl, —C₁₋₆alkenyl, —C₁₋₆alkynyl, —OR¹, —NR¹R², —C(═NR¹)NR²R³, —N(═NR¹)NR²R₃, —NR¹COR², —NR¹CO₂R², —NR¹SO₂R⁴, —NR¹CONR²R³, —SR⁴, —SOR⁴, —SO₂R⁴, —SO₂NR¹R², —COR¹, —CO₂R¹, —CONR¹R², —C(═NR¹)R², or —C(═NOR¹)R² substituents; wherein the alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R¹, R², and R³ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁴ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
B is phenyl —C₃₋₂₀cycloalkyl, —C₃₋₂₀cycloalkenyl, —C₃₋₂₀cycloalkadienyl, —C₃₋₂₀cycloalkatrienyl, —C₃₋₂₀cycloalkynyl, —C₃₋₂₀cycloalkadiynyl, indenyl, dihydroindenyl, naphthalenyl, dihydronaphthalenyl, pyridinyl, thiazolyl, furyl, dihydropyranyl, dihydrothiopyranyl, piperidinyl, isoxazolyl, pyridazinyl, pyrimidinyl, pyrazinyl, indolyl, quinolinyl, isoquinolinyl, optionally substituted with one to five independent halogen, —CN, NO₂, —C₁₋₆alkyl, —C₁₋₆alkenyl, —C₁₋₆alkynyl, —OR⁵, —NR⁵R⁶, —C(═NR⁵)NR⁶R⁷, —N(═NR⁵)NR⁶R⁷, —NR⁵COR⁶, —NR⁵CO₂R⁶, —NR⁵SO₂R⁸, —NR⁵CONR⁶R⁷, —SR⁸, —SOR⁸, —SO₂R⁸, —SO₂NR⁵R⁶, —COR⁵, —CO₂R⁵, —CONR⁵R⁶, —C(═NR⁵)R⁶, —C(═NOR⁵)R⁶, aryl or heterocycle substituents; wherein the alkyl, alkenyl or alkynyl may optionally be substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁵, R⁶, and R⁷ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁸ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents or a pharmaceutically acceptable salt thereof; and
wherein the compound is isotopically labeled with at least one ¹¹C, ¹³C, ¹⁴C, ¹⁸F, ¹⁵O, ¹³N, ³⁵S, ²H or ³H atom;
and except when A=6-methyl-2-pyridyl then B cannot be 3-methoxyphenyl or unsubstituted phenyl.

4. The compound of claim 2 wherein A is thiazolyl or isothiazolyl, optionally substituted with one to three independent halogen, —CN, NO₂, —C₁₋₆alkyl, —C₁₋₆alkenyl, —C₁₋₆alkynyl, —OR¹, —NR¹R², —C(═NR¹)NR²R³, —N(═NR¹)NR²R³, —NR¹COR², —NR¹CO₂R², —NR¹SO₂R⁴, —NR¹CONR²R³, —SR⁴, —SOR⁴, —SO₂R⁴, —SO₂NR¹R², —COR¹, —CO₂R¹, —CONR¹R², —C(═NR¹)R², or —C(═NOR¹)R² substituents; and
B is phenyl, —C₃₋₂₀cycloalkyl, —C₃₋₂₀cycloalkenyl, —C₃₋₂₀cycloalkadienyl, —C₃₋₂₀cycloalkatrienyl, —C₃₋₂₀cycloalkynyl, —C₃₋₂₀cycloalkadiynyl, indenyl, dihydroindenyl, naphthalenyl, dihydronaphthalenyl, pyridinyl, thiazolyl, furyl, dihydropyranyl, dihydrothiopyranyl, piperidinyl, isoxazolyl, pyridazinyl, pyrimidinyl, pyrazinyl, indolyl, quinolinyl, isoquinolinyl, optionally substituted with one to three independent halogen, —CN, NO₂, —C₁₋₆alkyl, —C₁₋₆alkenyl, —C₁₋₆alkynyl, —OR⁵, —NR⁵R⁶, —C(═NR⁵)NR⁶R⁷, —N(═NR⁵)NR⁶R⁷, —NR⁵COR⁶, —NR⁵CO₂R⁶, —NR⁵SO₂R⁸, —NR⁵CONR⁶R⁷, —SR⁸, —SOR⁸, —SO₂R⁸, —SO₂NR⁵R⁶, —COR⁵, —CO₂R⁵, —CONR⁵R⁶, —C(═NR⁵)R⁶, —C(═NOR⁵)R⁶, aryl or heterocycle substituents or a pharmaceutically acceptable salt thereof;
wherein the compound is isotopically labeled with at least one ¹¹C, ¹³C, ¹⁴C, ¹⁸F, ¹⁵O, ¹³N, ³⁵S, ²H, or ³H atom.

5. The compound of claim 1 wherein A is pyridinyl, pyrrolyl, imidazolyl, pyridazinyl, pyrimidinyl, pyrazoyl, pyrazinyl, triazolyl, triazinyl, tetrazolyl, tetrazinyl, tetrazepinyl, isoxazolyl, oxazolyl, oxadiazolyl, oxatriazolyl, oxazinyl, oxadiazinyl, isothiazolyl, thiazolyl, thiadazinyl, thiadiazolyl, thiadiazepinyl, dioxazolyl, oxathiazolyl, oxathiazinyl, oxazepinyl, oxadiazepinyl, azepinyl, and diazepinyl, optionally substituted with one to five independent halogen, —CN, NO₂, —C₁₋₆alkyl, —C₁₋₆alkenyl, —C₁₋₆alkynyl, —OR¹, —NR¹R², —C(═NR¹)NR²R₃, —N(═NR¹)NR²R₃, —NR¹COR², —NR¹CO₂R², —NR¹SO₂R⁴, —NR¹CONR²R³, —SR⁴, —SOR⁴, —SO₂R⁴, —SO₂NR¹R², —COR¹, —CO₂R¹, —CONR¹R², —C(═NR¹)R², or —C(═NOR¹)R² substituents;
R¹, R², and R³ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁴ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
B is pyridinyl or phenyl, optionally substituted with one to five independent halogen, —CN, NO₂, —C₁₋₆alkyl, —C₁₋₆alkenyl, —C₁₋₆alkynyl, —OR⁵, —NR⁵R⁶, —C(═NR⁵)NR⁶R⁷, —N(═NR⁵)NR⁶R⁷, —NR⁵COR⁶, —NR⁵CO₂R⁶, —NR⁵SO₂R⁸, —NR⁵CONR⁶R⁷, —SR⁸, —SOR⁸, —SO₂R⁸, —SO₂NR⁵R⁶, —COR⁵, —CO₂R⁵, —CONR⁵R⁶, —C(═NR⁵)R⁶, —C(═NOR⁵)R⁶, aryl or heterocycle substituents;
R⁵, R⁶, and R⁷ each independently is —C₀₋₆alkyl, —C₃₋₇cycloalkyl, heteroaryl, or aryl; any of which is optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents;
R⁸ is —C₁₋₆alkyl, —C₃₋₇cycloalkyl, heteroaryl, or aryl; optionally substituted with 1-5 independent halogen, —CN, —C₁₋₆alkyl, —O(C₀₋₆alkyl), —O(C₃₋₇cycloalkyl), —O(aryl), —N(C₀₋₆alkyl)(C₀₋₆alkyl), —N(C₀₋₆alkyl)(C₃₋₇cycloalkyl), —N(C₀₋₆alkyl)(aryl) substituents or a pharmaceutically acceptable salt thereof; and
wherein the compound is isotopically labeled with at least one ¹¹C, ¹³C, ¹⁴C, ¹⁸F, ¹⁵O, ¹³N, ³⁵S, ²H, or ³H atom;
and except when A=6-methyl-2-pyridyl then B cannot be 3-methoxyphenyl or unsubstituted phenyl.

6. The compound of claim 1 wherein A is selected from isothiazol-3-yl(1,2-thiazol-3-yl); thiazol-4-yl(1,3-thiazol-4-yl); thiazol-2-yl(1,3-thiazol-2-yl); oxazol-3-yl and oxazol-4-yl; 2-pyridinyl; 3-pyridinyl; 2-pyrrolyl; 3-pyridazinyl(1,2-diazin-3-yl); pyrimidin-4-yl(1,3-diazin-4-yl); pyrazin-3-yl(1,4-diazin-3-yl); pyrimidin-2-yl(1,3-diazin-2-yl); 1,3-isodiazol-4-yl; 1,3-isodiazol-2-yl; 1,2,3-triazin-4-yl; 1,2,4-triazin-6-yl; 1,2,4-triazin-3-yl; 1,2,4-triazin-5-yl; 1,3,5-triazin-2-yl; 1,2,3-triazol-4-yl; 1,2,4-triazol-3-yl; tetrazolyl; 1,2,4-thiadiazol-3-yl; 1,2,3-thiadiazol-4-yl; 1,3,4-thiadiazol-2-yl; 1,2,5-thiadiazol-3-yl; 1,2,4-thiadiazol-5-yl; 1,2,4-oxadiazol-3-yl; 1,2,3-oxadiazol-4-yl; 1,3,4-oxadiazol-2-yl; 1,2,5-oxadiazol-3-yl and 1,2,4-oxadiazol-5-yl.

7. The compound of claim 6, wherein A is thiazolyl or isothiazolyl.

8. The compound of claim 1 wherein B is a substituted or unsubstituted aryl, cycloalkyl, cycloalkenyl, cycloalkadienyl, cycloalkatrienyl, cycloalkynyl or cycloalkadiynyl, bicyclic hydrocarbon wherein two rings have two atoms in common, or a substituted or unsubstituted heterocycle, optionally containing one or more double bonds.

9. The compound of claim 8 wherein B is cyclopropanyl, cyclopentenyl and cyclohexenyl, indenyl, dihydroindenyl, phenyl, naphthalenyl dihydronaphthalenyl, thiazolyl, furyl, dihydropyranyl, dihydrothiopyranyl, piperidinyl, isoxazolyl, pyridinyl, pyridazinyl, pyrimidinyl, pyrazinyl, indolyl and isoquinolinyl.

10. The compound of claim 9, wherein B is pyridinyl or phenyl.

11. An isotopically labeled compound selected from: 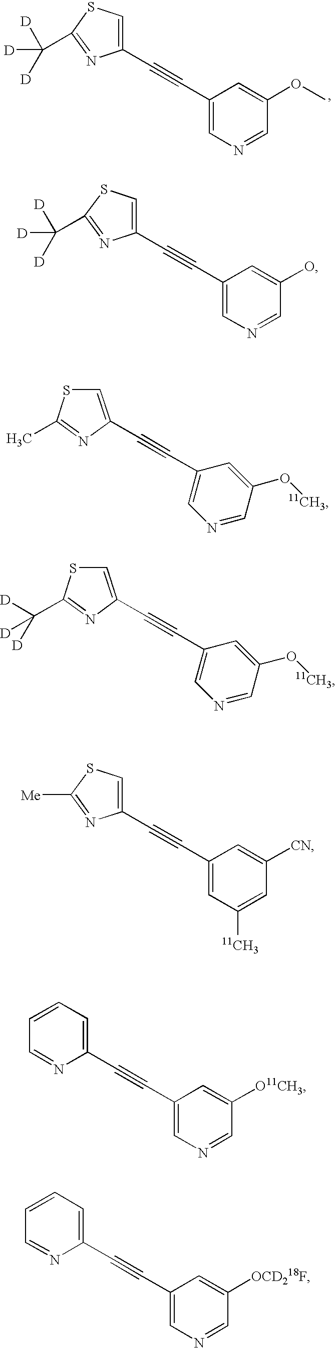 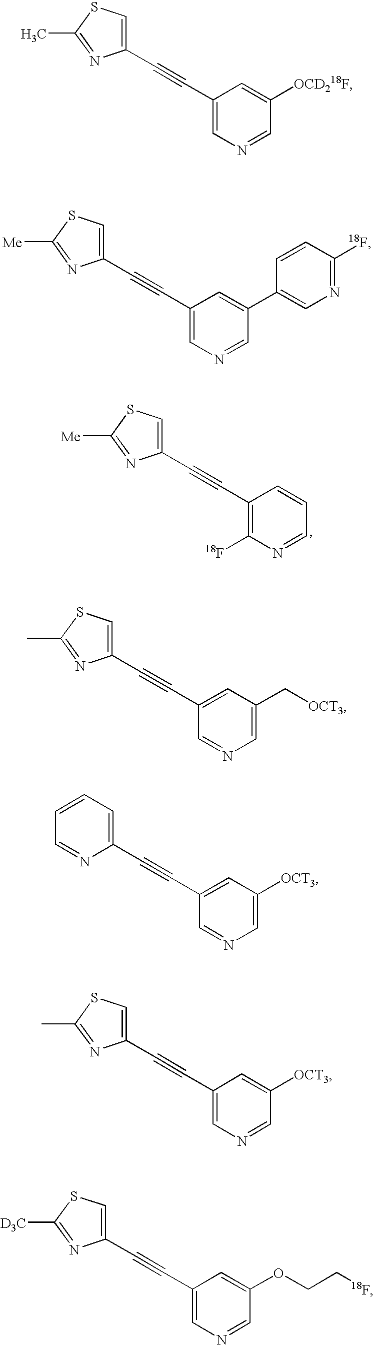 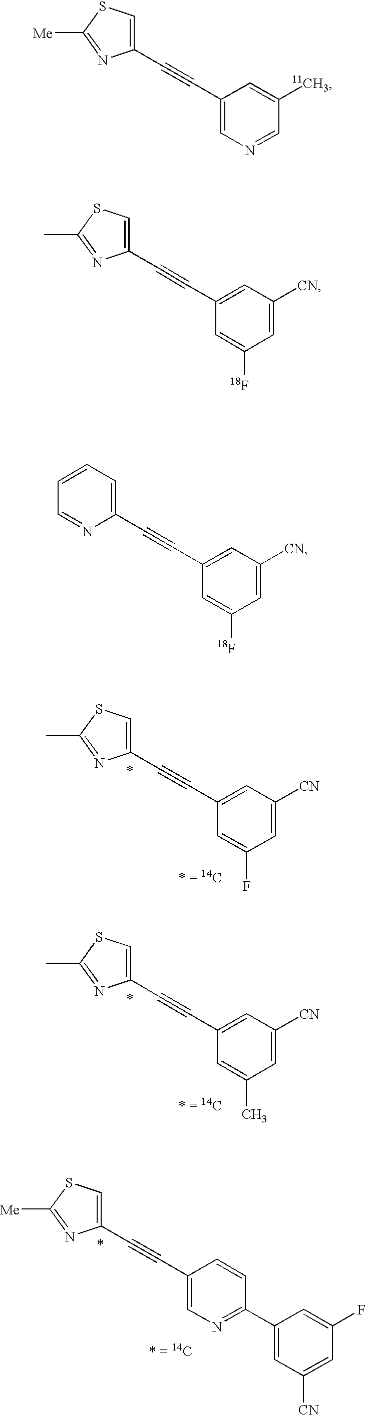 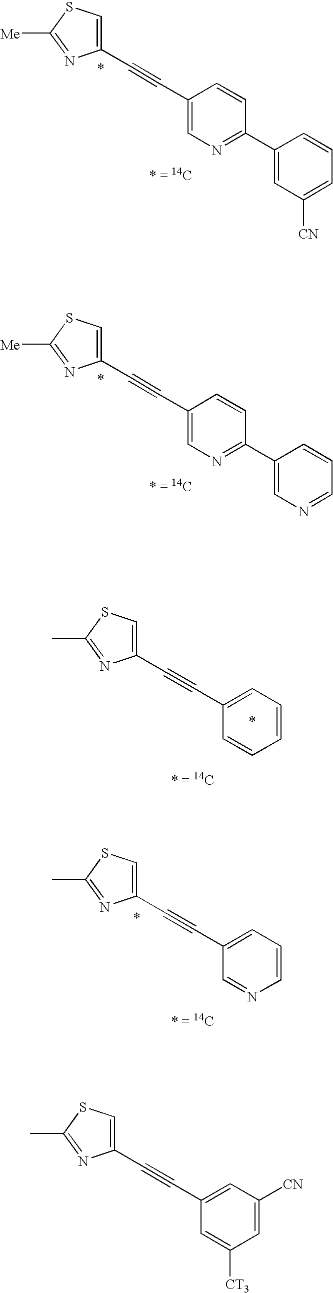
or a pharmaceutically acceptable salt thereof.

12. A method for the preparation of the isotopically labeled compounds according to claim 1 comprising the steps of reacting a precursor of a compound of claim 1 with an isotopically labeled reagent containing one or more isotopes selected from ¹¹C, ¹³C, ¹⁴C ¹⁸F, ¹⁵O, ¹³N, ³⁵S, ²H, and ³H which is capable of reacting with said precursor wherein said isotopically labeled reagent produces an isotopically labeled substituent on said substrate using standard organic synthetic chemistry procedures to produce a compound of claim 1.

13. A method of performing positron emission tomography (PET) imaging comprising a step of administering a compound according to claim 1 as a tracer compound

14. A method of performing positron emission tomography (PET) imaging comprising a step of administering a compound according to claim 5 as a tracer compound.

15. A method for imaging metabotropic glutamate receptors in a metabotropic glutamate receptor-rich tissue comprising:
a) administering an effective quantity of an isotopically labeled metabotropic glutamate receptor ligand according to claim 1;
b) positioning the subject in a PET device;
c) performing the emission scan of the metabotropic glutamate receptor-rich tissue, and obtaining a PET image of the tissue; and
d) evaluating the PET image for the presence or absence of focally increased uptake of the isotopically labeled ligand in the tissue.

16. A method for imaging metabotropic glutamate receptors in a metabotropic glutamate receptor-rich tissue comprising:
a) administering an effective quantity of an isotopically labeled metabotropic glutamate receptor ligand according to claim 5;
b) positioning the subject in a PET device;
c) performing the emission scan of the metabotropic glutamate receptor-rich tissue, and obtaining a PET image of the tissue; and evaluating the PET image for the presence or absence of focally increased uptake of the isotopically labeled ligand in the tissue.

17. The method of claim 15 wherein the metabotropic glutamate receptor-rich tissue is cerebral tissue or neurotissue.

18. The method in claim 15 where the tracer in the PET imaging allows monitoring of the metabolic activity of metabotropic receptors in vivo.

19. A method for diagnosing and monitoring the treatment of metabotropic glutamate receptor-modulated conditions, diseases or disorders comprising a step of administering to a patient suspected of having said condition, disease, or disorder an effective tracer amount of the compound of claim 11.

20. An isotopically labeled compound of Formula III wherein X is —¹¹CH₃ or ¹⁸F and Y is H or ²H: 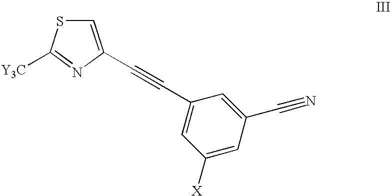
or a pharmaceutically acceptable salt thereof.

21. A method of performing positron emission tomography (PET) imaging to determine the receptor occupancy of a mGluR5 agonist or antagonist comprising a step of administering a compound according to claim 1 as a tracer.

22. A method of using an isotopically labeled compound to determine the receptor occupancy of a mGluR5 agonist or antagonist comprising a step of administering a compound according to claim 1 as a tracer.
